(19) United States
(12) Patent Application Publication
(10) Pub. No.: US 2005/0136035 A1
(43) Pub. Date: Jun. 23, 2005

(54) Cell specific replication-competent viral vectors comprising a self processing peptide cleavage site

(76) Inventors: Derek Ko, San Francisco, CA (US); Yuanhao Li, Palo Alto, CA (US); Thomas Harding, San Francisco, CA (US); Jianmin Fang, Palo Alto, CA (US); Nagarajan Ramesh, Sunnyvale, CA (US); De Chao Yu, Palo Alto, CA (US)

Correspondence Address: Supervisor, Patent Prosecution Services;PIPER RUDNICK LLP, 1200 Nineteenth Street, N.W., Washington, DC, 20036-2412, US

(21) Appl. No.: 10/857,498

(22) Filed: Jun. 1, 2004

Related U.S. Application Data
(60) Provisional application No. 60/475,005, filed on Jun. 3, 2003.

(51) Int. Cl. A61K 48/00; C12N 15/861; C12N 5/08

(52) U.S. Cl. 424/93.2; 435/366; 435/456

(57) ABSTRACT

Cell specific replication-competent viral vectors comprising a self processing peptide cleavage sequence are provided. The targeted replication-competent viral vectors include two or more co-transcribed genes under transcriptional control of the same heterologous transcriptional regulatory element (TRE), wherein at least a second gene is under translational control of a self processing cleavage sequence or 2A sequence. Exemplary vector constructs may further include an additional proteolytic cleavage site which provides a means to remove the self processing peptide sequence from the viral vector.

This application claims priority from U.S. Provisional Application Ser. No. 60/475,005 filed Jun. 3, 2003. The entirety of that provisional application is incorporated herein by reference.

### BACKGROUND OF THE INVENTION

1. Field of the Invention

The invention relates to targeted replication-competent viral vectors which include two or more co-transcribed genes under transcriptional control of a heterologous transcriptional regulatory element (TRE), wherein at least a second gene is under translational control of a self processing cleavage sequence or 2A sequence.

2. Background of the Technology

To express two or more proteins from a single viral or non-viral vector, an individual promoter for each protein or an internal ribosome entry site (IRES) sequence is commonly used to drive expression of the coding sequence for the respective proteins. If two genes are linked via an IRES sequence the expression level of the second gene may be significantly reduced (Furler et al., Gene Therapy 8:864-873 (2001)).

Replication-competent viral vectors, which take advantage of the cytotoxic effects associated with virus replication, are currently in use as agents for cancer therapy. Such replication-competent viral vectors, also termed “oncolytic vectors” typically comprise a gene essential for viral replication under control of a transcriptional regulatory element (TRE), thus limiting viral replication to cells in which the TRE is functional.

At present internal ribosome entry sites (IRES) typically serve as a way to place two or more viral genes under the control of a specific promoter without the need for additional TREs (Li, Y et al., Cancer Research, 2001; 17: 6428-6436; Zhang, J et al., Cancer Research, 2002; 13: 3743-3750). See also, WO01/73093 which describes cell-specific adenovirus vectors comprising an internal ribosome entry site. The use of an IRES to control transcription of two or more genes operably linked to the same promoter can result in lower level expression of the second, third, etc. gene relative to the gene adjacent the promoter. In addition, an IRES sequence may be sufficiently long to present issues with the packaging limit of the vector, e.g., the ECMV IRES has a length of 507 base pairs.

The linking of proteins in the form of polyproteins is a strategy adopted in the replication of many viruses including picornaviridae. Upon translation, virus-encoded peptides mediate rapid intramolecular (cis) cleavage of a polyprotein to yield discrete mature protein products. Foot and Mouth Disease viruses (FMDV) are a group within the picornaviridae which express a single, long open reading frame encoding a polyprotein of approximately 225 kD. The full length translation product undergoes rapid intramolecular (cis) cleavage at the C-terminus of a 2A region occurring between the capsid protein precursor (P1-2A) and replicative domains of the polyprotein 2BC and P3, and this cleavage is mediated by proteinase-like activity of the 2A region itself (Ryan et al., J. Gen. Virol. 72:2727-2732 (1991); Vakharia et al., J. Virol. 61:3199-3207 (1987)). Constructs including the essential amino acid residues for expression of the cleavage activity by the FMDV 2A region have been designed (Ryan, 1991). 2A domains have also been characterized from aphthoviridea and cardioviridae of the picornavirus family (Donnelly et al., J. Gen. Virol. 78:13-21 (1997).

The Foot and Mouth Disease Virus 2A sequence is a small peptide (approximately 18 amino acids in length) that has been shown to mediate the ‘cleavage’ of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

The 2A sequence provides the advantages of both a reduced size together with the ability to facilitate expression of two or more genes from the same promoter in essentially equimolar amounts. A direct comparison of the expression of two or more genes mediated by the 2A sequence relative to the ECMV IRES indicated that secondary genes are expressed at higher levels in cassettes employing the 2A sequence as compared to the ECMV IRES (Furler, S et al. Gene Therapy, 2001; 8: 864-873).

First generation oncolytic viruses rely on cell type or cell status-specific regulatory elements to limit viral replication to specific cell types, i.e., cancer cells. However, the use of two or more cell type or cell status-specific regulatory elements to control expression of viral and/or therapeutic genes is likely to result in greater specificity of viral replication and greater killing of target cells such as cancer cells. The need for controlled expression of two or more gene products together with the packaging limitations of viral vectors such as adenovirus, limits the choices with respect to vector construction. Furthermore, the use of two promoters within a single vector can result in promoter interference causing inefficient expression of both genes.

Accordingly, there remains a need for improved gene expression systems in the context of replication competent viral vectors which correct for the deficiencies inherent in currently available technology (e.g., the use of an IRES). The present invention addresses this need in the context of oncolytic viruses.

### SUMMARY OF THE INVENTION

The present invention provides improved replication competent viral vectors comprising two or more co-transcribed genes under transcriptional control of a heterologous transcriptional regulatory element (TRE), wherein at least a second gene is under translational control of a self processing cleavage site. In one embodiment, the first and second viral genes are co-transcribed as a single mRNA and the second gene is not operably linked to a promoter, but is under translational control of a self-processing cleavage site. In one aspect of this embodiment, the first and second genes are viral genes essential for viral replication. In another aspect, the first gene is a viral gene and the second gene is a therapeutic gene.

In one exemplary embodiment, the invention provides replication competent adenoviral vectors which include an essential adenoviral gene under transcriptional control of a heterologous transcriptional regulatory element (TRE), wherein the essential gene is an adenoviral early gene, for example, E1A, E1B, or E4, or an adenoviral late gene and the vector further includes at least a second gene under translational control of a self processing cleavage site.

In one aspect of this embodiment, the first adenoviral gene is E1A, and the second adenoviral gene is E1B. Optionally, the endogenous promoter for one or more of the co-transcribed adenovirus genes essential for replication, e.g., E1A, is deleted and/or mutated such that the gene is under sole transcriptional control of the heterologous TRE.

In another aspect, the invention provides adenovirus vectors comprising an adenovirus gene essential for viral replication under control of a heterologous TRE, wherein the adenovirus gene is E1A, the native (endogenous) E1A promoter is deleted and the vector further comprises at least a second gene under translational control of a self processing cleavage site.

In a related aspect, the adenovirus gene is E1B wherein the native (endogenous) E1B promoter is deleted, the E1B gene is under transcriptional control of a heterologous cell-specific TRE and the vector further comprises at least a second gene under translational control of a self processing cleavage site.

In other embodiments, an enhancer element for first and/or second adenovirus genes is inactivated or the adenovirus vector comprises a TRE which has its endogenous silencer element inactivated.

Any TRE which directs cell-specific expression can be used in the disclosed vectors. In some embodiments, the target cell-specific TRE is a cell status-specific TRE. In yet other embodiments, the target cell-specific TRE is a tissue specific TRE. Exemplary TREs include, but are not limited to, TREs specific for prostate cancer cells, breast cancer cells, hepatoma cells, melanoma cells, bladder cells and/or colon cancer cells. Exemplary TREs include, but are not limited to a cell type-specific TRE (e.g., a probasin (PB); a prostate-specific antigen (PSA) TRE comprising a PSA-specific promoter and/or a PSA-specific enhancer; an alpha-fetoprotein (AFP) TRE; a human kallikrein (hKLK2) TRE; a tyrosinase TRE; a human uroplakin II (hUPII) TRE; a carcinoembryonic antigen (CEA) TRE; a melanocyte-specific TRE comprising a melanocyte-specific promoter and/or a melanocyte-specific enhancer; a HER-2/neu TRE; a liver-specific CRG-L2 TRE; a PRL-3 TRE; a mucin (MUC1) TRE); or a cell status TRE (e.g., an E2F TRE, an H19 TRE, or a telomerase (TERT) TRE).

Preferred self-processing cleavage sites include a 2A sequence, e.g., a 2A sequence derived from Foot and Mouth Disease Virus (FMDV).

In a further preferred aspect, the vector comprises a sequence which encodes an additional proteolytic cleavage site located between the first gene and second genes, e.g., a furin cleavage site with the consensus sequence RXK(R)R (presented as SEQ ID NO:10).

The present invention also provides viral vectors which comprise a therapeutic gene or coding sequence (also termed “transgene”), for example, a cytotoxic gene or the coding sequence for a cytokine. The therapeutic gene may be under the transcriptional control of the same TRE as a first viral gene or under the transcriptional control of the same TRE as a first viral gene and a second viral gene and may or may not be under translational control of a 2A sequence.

In addition, the present invention provides compositions and host cells comprising a replication-competent viral vector and a pharmaceutically acceptable excipient. Host cells include those used for propagation of a vector and those into which the vector is introduced for therapeutic purposes.

In another aspect, methods are provided for propagating replication-competent viral vectors specific for mammalian cells which permit the function of a heterologous TRE, wherein the method comprises combining a viral vector of the invention with mammalian cells that permit the function of the heterologous TRE, such that the viral vector(s) enters the cell and the virus is propagated.

In another aspect, methods are provided for conferring selective cytotoxicity on target cells, by contacting the cells with a replication-competent viral vector of the invention whereby the vector enters the cell and replicates therein, resulting in cytoxicity to the cells.

The invention further provides methods of suppressing tumor cell growth, e.g., of a target tumor cell, comprising contacting the tumor cell with a replication-competent viral vector of the invention such that the vector enters the tumor cell and exhibits selective cytotoxicity for the cell.

In yet another aspect, methods are provided for modifying the genotype of a target cell, comprising contacting the cell with a replication-competent viral vector of the invention, wherein the viral vector enters the cell and replicates therein.

### DESCRIPTION OF THE FIGURES

FIGS. 1A-I provide schematic depictions of exemplary constructs: 2A element in a bicistronic cassette expressing E1A and E1B under the control of the E2F promoter (FIG. 1A); 2A element in a bicistronic cassette expressing E1A and GM-CSF under the control of the E2F promoter (FIG. 1B); 2A element in a bicistronic cassette expressing E1B55K and GM-CSF under the control of the telomerase promoter (FIG. 1C); 2A element in a bicistronic cassette expressing fiber and GM-CSF under the control of the adenoviral major late promoter (FIG. 1D); 2A element in a bicistronic cassette in which CD and GM-CSF are expressed, by alternative splicing, under the control of the endogenous E3 promoter (FIG. 1E); 2A element in a bicistronic cassette expressing E4 ORF 1 and GM-CSF, followed by E4 ORF 2-7, under the control of the endogenous E4 promoter (FIG. 1F); 2A element in a bicistronic cassette expressing penton and GM-CSF under the control of the major late promoter (FIG. 1G); 2A element in a bicistronic cassette expressing hexon and GMCSF under the control of the major late promoter (FIG. 1H); and 2A element in a bicistronic cassette expressing E2A and GM-CSF, followed by E2B, under the control of the endogenous E2 promoter (FIG. 1I).

FIGS. 2A-E provide schematic depictions of exemplary E2F-controlled vectors featuring E1A-FMDV 2A-E1B 55k cassettes with various modifications to the E1A/E1B junction and the E1B region, wherein FIG. 2A is a schematic depiction of OV1054.11 (M.2.2.C); FIG. 2B is a schematic depiction of OV1057; FIG. 2C is a schematic depiction of OV945; FIG. 2D is a schematic depiction of OV1056; FIG. 2E is a schematic depiction of OV1054.11. (M.1.1.B); and FIG. 2F is a schematic depiction of OV802 (wt Ad5).

FIGS. 3A-C illustrate the results of PCR analysis of OV802, OV1054.11.M.2.2.C and OV1057 vectors following multiple passages of each vector through A549 cells, using primers specific for sequences in the wild-type adenovirus 5 genome (FIG. 3A), and following treatment with restriction enzymes, BstX I and Acc I, respectively (FIGS. 3B and 3C).

FIG. 4 illustrates the results of a virus yield assay using a panel of both tumor and normal cell lines to test viral production by OV802, OV945, OV1056, OV1057 and OV1054 vectors. The results are reported as PFU/cell and are summarized in Table 3.

FIGS. 5A and B depict the results of Western blot analysis for the expression of adenoviral proteins by A549 cells infected with OV945, OV1057, OV1054.11.M.2.2.C, OV 1056, OV1054.11.M.1.1.B and OV802 vectors, respectively, performed using antibodies to E1A (FIG. 5A) and E1B 55k (FIG. 5B).

FIGS. 6A-C illustrate the results of MTS cytotoxicity assays following infection with OV945, OV802, OV1057, OV1054.11.M.2.2.C, OV1054.11.M.1.1.B and OV 1056, vectors, respectively, in SW 780 cells (FIG. 6A), Panc I cells (FIG. 6B), LoVo cells (FIG. 6C) and MRC5 cells (FIG. 6D).

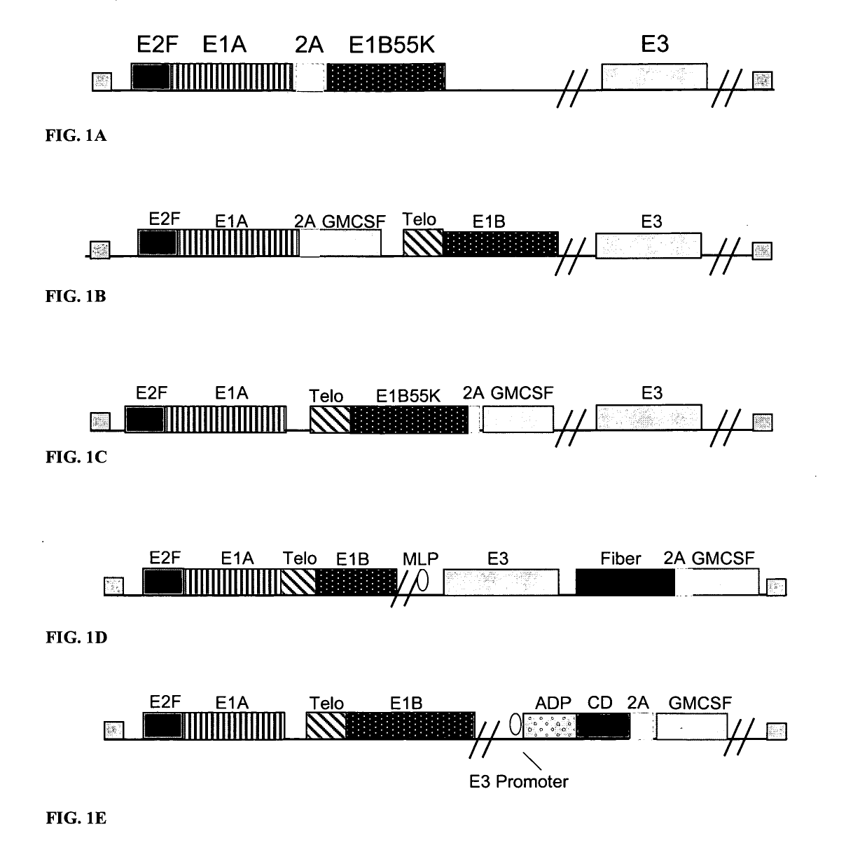

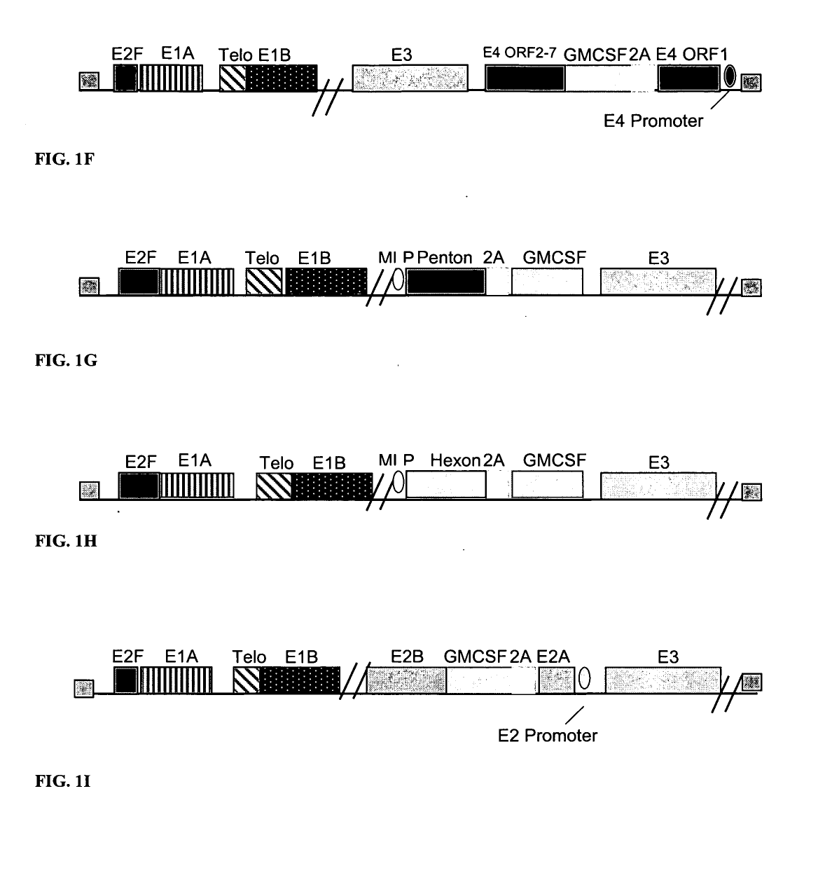

### DETAILED DESCRIPTION OF THE INVENTION

Definitions

Unless otherwise indicated, all terms used herein have the same meaning as they would to one skilled in the art and the practice of the present invention will employ, conventional techniques of microbiology and recombinant DNA technology, which are within the knowledge of those of skill of the art.

The term “vector”, as used herein, refers to a DNA or RNA molecule such as a plasmid, virus or other vehicle, which contains one or more heterologous or recombinant DNA sequences and is designed for transfer between different host cells. The terms “expression vector” and “gene therapy vector” refer to any vector that is effective to incorporate and express heterologous DNA fragments in a cell. A cloning or expression vector may comprise additional elements, for example, the expression vector may have two replication systems, thus allowing it to be maintained in two organisms, for example in human cells for expression and in a prokaryotic host for cloning and amplification. Any suitable vector can be employed that is effective for introduction of nucleic acids into cells such that protein or polypeptide expression results, e.g. a viral vector or non-viral plasmid vector. Any cells effective for expression, e.g., insect cells and eukaryotic cells such as yeast or mammalian cells are useful in practicing the invention.

A “viral construct” or “viral vector”, e.g., an “adenovirus construct” or “adenoviral vector” is a polynucleotide construct which may take any of several forms, including, but not limited to, DNA, DNA encapsulated in simplex, DNA encapsulated in liposomes, DNA complexed with polylysine, complexed with synthetic polycationic molecules, conjugated with transferrin, and complexed with compounds such as PEG to immunologically “mask” the molecule and/or increase half-life, and conjugated to a nonviral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides. For purposes of this invention, viral vectors are replication-competent in a target cell.

A “replication competent viral vector” refers to a polynucleotide construct of viral origin that can replicate in the absence of complementing helper genes.

The term “replication-competent” as used herein relative to the viral vectors of the invention generally refers to adenoviral vectors and particles that preferentially replicate in certain types of cells or tissues but to a lesser degree or not at all in other types. In one embodiment of the invention, the adenoviral vector and/or particle selectively replicates in tumor cells and or abnormally proliferating tissue, such as solid tumors and other neoplasms. These include the viruses disclosed in U.S. Pat. Nos. 5,677,178, 5,698,443, 5,871,726, 5,801,029, 5,998,205, and 6,432,700, the disclosures of which are incorporated herein by reference in their entirety. Such viruses may be referred to as “oncolytic viruses” or “oncolytic vectors” and may be considered to be “cytolytic” or “cytopathic” and to effect “selective cytolysis” of target cells.

The terms “heterologous DNA” and “heterologous RNA” refer to nucleotides that are not endogenous (native) to the cell or part of the genome in which they are present. Generally heterologous DNA or RNA is added to a cell by transduction, infection, transfection, transformation or the like, as further described below. Such nucleotides generally include at least one coding sequence, but the coding sequence need not be expressed. The term “heterologous DNA” may refer to a “heterologous coding sequence” or a “transgene”.

As used herein, the term “gene” or “coding sequence” means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

The term “operably linked” as used herein relative to a recombinant DNA construct or vector means nucleotide components of the recombinant DNA construct or vector are functionally related to one another for operative control of a selected coding sequence. Generally, “operably linked” DNA sequences are contiguous, and, in the case of a secretory leader, contiguous and in reading frame. However, enhancers do not have to be contiguous.

A “promoter” is a DNA sequence that directs the binding of RNA polymerase and thereby promotes RNA synthesis, i.e., a minimal sequence sufficient to direct transcription. Promoters and corresponding protein or polypeptide expression may be cell-type specific, tissue-specific, or species specific. Also included in the nucleic acid constructs or vectors of the invention are enhancer sequences which may or may not be contiguous with the promoter sequence. Enhancer sequences influence promoter-dependent gene expression and may be located in the 5′ or 3′ regions of the native gene.

“Enhancers” are cis-acting elements that stimulate or inhibit transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region.

A “regulatable promoter” is any promoter whose activity is affected by a cis or trans acting factor (e.g., an inducible promoter, such as an external signal or agent).

A “constitutive promoter” is any promoter that directs RNA production in many or all tissue/cell types at most times, e.g., the human CMV immediate early enhancer/promoter region which promotes constitutive expression of cloned DNA inserts in mammalian cells.

The terms “transcriptional regulatory protein”, “transcriptional regulatory factor” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA may be indirect by way of binding to another protein that in turn binds to, or is bound to a DNA response element.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson R J, Howell M T, Kaminski A (1990) Trends Biochem Sci 15(12):477-83) and Jackson R J and Kaminski, A. (1995) RNA 1(10):985-1000. The examples described herein are relevant to the use of any IRES element, which is able to promote direct internal ribosome entry to the initiation codon of a cistron. “Under translational control of an IRES” as used herein means that translation is associated with the IRES and proceeds in a cap-independent manner.

A “self-processing cleavage site” or “self-processing cleavage sequence” is defined herein as a post-translational or co-translational processing cleavage site or sequence. Such a “self-processing cleavage” site or sequence refers to a DNA or amino acid sequence, exemplified herein by a 2A site, sequence, or domain or a 2A-like site, sequence, or domain. As used herein, a “self-processing peptide” is defined herein as the peptide expression product of a DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

A “multicistronic transcript” refers to an mRNA molecule which contains more than one protein coding region, or cistron. An mRNA comprising two coding regions is denoted a “bicistronic transcript.” The “5′-proximal” coding region or cistron is the coding region with a translation initiation codon (usually AUG) is closest to the 5′-end of a multicistronic mRNA molecule. A “5′-distal” coding region or cistron is one whose translation initiation codon (usually AUG) is not the closest initiation codon to the 5′ end of the mRNA. The terms “5′-distal” and “downstream” are used synonymously to refer to coding regions that are not adjacent to the 5′ end of an mRNA molecule.

As used herein, “co-transcribed” means that two (or more) polynucleotide coding regions are under transcriptional control of a single transcriptional control element.

As used herein, the term “additional proteolytic cleavage site”, refers to a sequence which is incorporated into an expression construct of the invention adjacent a self-processing cleavage site, such as a 2A or 2A like sequence, and provides a means to remove additional amino acids that remain following cleavage by the self processing cleavage sequence. Exemplary “additional proteolytic cleavage sites” are described herein and include, but are not limited to, furin cleavage sites with the consensus sequence RXK(R)R (SEQ ID NO:10). Such furin cleavage sites can be cleaved by endogenous subtilisin-like proteases, such as furin and other serine proteases within the protein secretion pathway.

As used herein, a “transcription response element” or “transcriptional regulatory element”, or “TRE” is a polynucleotide sequence, preferably a DNA sequence, which increases transcription of an operably linked polynucleotide sequence in a host cell that allows that TRE to function. A TRE can comprise an enhancer and/or a promoter. A “transcriptional regulatory sequence” is a TRE. A “target cell-specific transcriptional response element” or “target cell-specific TRE” is a polynucleotide sequence, preferably a DNA sequence, which is preferentially functional in a specific type of cell, that is, a target cell. Accordingly, a target cell-specific TRE transcribes an operably linked polynucleotide sequence in a target cell that allows the target cell-specific TRE to function.

The terms “target cell-specific”, “tumor cell-specific” and “cell status-specific” as used herein, are intended to include cell type specificity, tissue specificity, developmental stage specificity, and tumor specificity, as well as specificity for a cancerous state of a given target cell. A “target cell-specific TRE” may be a cell type-specific or cell status-specific TRE, or a “composite” TRE. The term “composite TRE” includes a TRE which comprises both a cell type-specific and a cell status-specific TRE. A target cell-specific TRE can also include a heterologous component, including, for example, an SV40 or a cytomegalovirus (CMV) promoter. By specific is meant the TRE is preferentially functional, i.e., confers transcriptional activation on an operably linked polynucleotide in a cell which allows the TRE to function. It will be understood that such “specificity” need not be absolute, but requires preferential replication in a target cell (as further defined herein below).

As used herein, the term “cell status-specific TRE” is preferentially functional, i.e., confers transcriptional activation on an operably linked polynucleotide in a cell which allows a cell status-specific TRE to function, such as a cell which exhibits a particular physiological condition, including, but not limited to, an aberrant physiological state. “Cell status” thus refers to a given, or particular, physiological state (or condition) of a cell, which is reversible and/or progressive. The physiological state may be generated internally or externally; for example, it may be a metabolic state (such as in response to conditions of low oxygen), or it may be generated due to heat or ionizing radiation). In some embodiments, in accordance with cell status, the TRE is functional in or during: low oxygen conditions (hypoxia); certain stages of the cell cycle, such as S phase; elevated temperature; ionizing radiation. Adenovirus vectors containing cell status-specific response elements are described in WO00/15820, expressly incorporated by reference herein. In other words, “cell status” is distinct from “cell type”, which relates to a differentiation state of a cell, which under normal conditions is irreversible.

A “functional portion” of a target cell-specific TRE is one which confers target cell-specific transcription on an operably linked gene or coding region, such that the operably linked gene or coding region is preferentially expressed in target cells.

By “transcriptional activation” or an “increase in transcription,” it is intended that transcription is increased above basal levels in a target cell by at least about 2 fold, preferably at least about 5 fold, preferably at least about 10 fold, more preferably at least about 20 fold, more preferably at least about 50 fold, more preferably at least about 100 fold, more preferably at least about 200 fold, even more preferably at least about 400 fold to about 500 fold, even more preferably at least about 1000 fold. Basal levels are generally the level of activity (if any) in a non-target cell (i.e., a different cell type), or the level of activity (if any) of a reporter construct lacking a target cell-specific TRE as tested in a target cell line.

“Transformation” is typically used to refer to bacteria comprising heterologous DNA or cells which express an oncogene and have therefore been converted into a continuous growth mode such as tumor cells. A vector used to “transform” a cell may be a plasmid, virus or other vehicle.

Typically, a cell is referred to as “transduced”, “infected”, “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

As used herein, the terms “stably transformed”, “stably transfected” and “transgenic” refer to cells that have a non-native (heterologous) nucleic acid sequence integrated into the genome. Stable transfection is demonstrated by the establishment of cell lines or clones comprised of a population of daughter cells containing the transfected DNA stably integrated into their genomes. In some cases, “transfection” is not stable, i.e., it is transient. In the case of transient transfection, the exogenous or heterologous DNA is expressed, however, the introduced sequence is not integrated into the genome and is considered to be episomal.

A “host cell” includes an individual cell or cell culture which can be or has been a recipient of a viral vector(s) of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with an adenoviral vector of this invention.

As used herein, the terms “protein” and “polypeptide” may be used interchangeably and typically refer to “proteins” and “polypeptides” of interest that are expressed using the self processing cleavage site-containing vectors of the present invention. Such “proteins” and “polypeptides” may be any protein or polypeptide useful for research, diagnostic or therapeutic purposes, as further described below.

“Replication” and “propagation” are used interchangeably and refer to the ability of a viral vector of the invention to reproduce or proliferate. These terms are well understood in the art. For purposes of this invention, replication involves production of viral proteins, e.g. adenoviral proteins and is generally directed to reproduction of virus. Replication can be measured using assays standard in the art and described herein, such as a burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

“Replicating preferentially” or “preferential replication”, as used herein, means that the viral vector replicates more in a target cell than a non-target cell. By “targeted” with respect to a replication-competent viral vector, it is meant that the viral vector replicates preferentially in a target cell relative to a non-target cell. Preferably, the viral vector replicates at a significantly higher rate in target cells than non target cells; preferably, at least about 2-fold higher, preferably, at least about 5-fold higher, more preferably, at least about 10-fold higher, still more preferably at least about 50-fold higher, even more preferably at least about 100-fold higher, still more preferably at least about 400- to 500-fold higher, still more preferably at least about 1000-fold higher, most preferably at least about 1×10⁶ higher. Most preferably, the virus replicates solely in the target cells (that is, does not replicate or replicates at a very low levels in non-target cells).

“Under transcriptional control” is a term well understood in the art and indicates that transcription of a polynucleotide sequence, usually a DNA sequence, depends on its being operably (operatively) linked to an element which contributes to the initiation of, or promotes, transcription.

An “E3 region” (used interchangeably with “E3”) is a term well understood in the art and means the region of the adenoviral genome that encodes the E3 products (discussed herein). Generally, the E3 region is located between about 28583 and 30470 of the adenoviral genome. The E3 region has been described in various publications, including, for example, Wold et al. (1995) Curr. Topics Microbiol. Immunol. 199:237-274. In some embodiments, a recombinant adenoviral vector of the invention comprises a mutation or deletion in an E3 coding region, such as E3-6.7, KDa, gp19KDa, 11.6 KDa (ADP), 10.4 KDa (RIDα), 14.5 KDa (RIDβ), and E3-14.7 Kda or a deletion in the E1b gene such as a deletion in the gene which encodes the E1b 19 kD protein, e.g. the deletion presented as SEQ ID NO:12. In other embodiments, a recombinant adenoviral vector of the invention comprises a the coding sequence for at least one native E3 protein by providing a vector including an E3 coding region, selected from E3-6.7, KDa, gp19KDa, 11.6 KDa (ADP), 10.4 KDa (RIDα), 14.5 KDa (RIDβ), and E3-14.7 Kda.

A “portion” of the E3 region means less than the entire E3 region, and as such includes polynucleotide deletions as well as polynucleotides encoding one or more polypeptide products of the E3 region.

An “E1B 19-kDa region” (used interchangeably with “E1B 19-kDa genomic region”) refers to the genomic region of the adenovirus E1B gene encoding the E1B 19-kDa product. According to wild-type Ad5, the E1B 19-kDa region is a 261 bp region located between nucleotide 1714 and nucleotide 2244. The E1B 19-kDa region has been described in, for example, Rao et al., Proc. Natl. Acad. Sci. USA, 89:7742-7746. The present invention encompasses deletion of all or part of the E 1 B 19-kDa region as well as embodiments wherein the E 1 B 19-kDa region is mutated.

Compositions and Methods of The Invention

The invention provides replication-competent viral vectors incorporating a self-processing cleavage site or sequence in bicistronic or multicistronic cassettes expressing viral genes essential for replication, and/or therapeutic genes. These vectors provide the advantage of enhanced expression of two or more genes under transcriptional control of the same promoter as well as allowing for more equal expression of the two or more genes than is typically obtained using an IRES. The use of a self-processing cleavage site also eliminates the need for a separate promoter for each gene, thereby eliminating the possibility of promoter interference. The self-processing cleavage site-containing replication-competent viral vectors of the invention can also be used for target cell-specific delivery of the vectors, in particular to cancer cells.

Accordingly, the invention described herein provides an improved replication-competent viral vector system containing a self-processing cleavage site, exemplified herein by a 2A or 2A-like sequence. This improved replication-competent viral vector system provides the opportunity to express two or more genes under transcriptional control of a single promoter such that the proteins are cleaved apart co-translationally with high efficiency. This strategy for expression of self-processing proteins/polypeptides/peptides is readily applicable to replication-competent viral vector systems and methods of using the same.

The two or more genes under transcriptional control of the same promoter may be adenoviral genes or heterologous genes (transgenes) and the promoter may be a native adenoviral promoter or a heterologous promoter (which is constitutive, inducible, cell type, cell status or tissue specific).

Transcriptional Regulatory Elements (TREs)

Cell- and tissue-specific transcriptional regulatory element (TREs), as well as methods for their identification, isolation, characterization, genetic manipulation and use for regulation of operatively linked coding sequences, are known in the art.

A TRE can be derived from the transcriptional regulatory sequence of a single gene, sequences from different genes can be combined to produce a functional TRE, or a TRE can be synthetically generated (e.g. the CTP4 promoter). A TRE can be tissue-specific, tumor-specific, developmental stage-specific, cell status specific, etc., depending on the type of cell present in the tissue or tumor. Such TREs may be collectively referred to herein as tissue-specific or target cell-specific. As described in more detail herein, a target cell-specific TRE can comprise any number of configurations, including, but not limited to, a target cell-specific promoter and target cell-specific enhancer; a heterologous promoter and a target cell-specific enhancer; a target cell-specific promoter and a heterologous enhancer; a heterologous promoter and a heterologous enhancer; and multimers of the foregoing. The promoter and enhancer components of a target cell-specific TRE may be in any orientation and/or distance from the coding sequence of interest, as long as the desired target cell-specific transcriptional activity is obtained. Transcriptional activation can be measured in a number of ways known in the art (and described in more detail below), but is generally measured by detection and/or quantitation of mRNA or the protein product of the coding sequence under control of (i.e., operably linked to) the target cell-specific TRE.

As further discussed herein, a target cell-specific TRE can be of varying lengths, and of varying sequence composition. A target cell-specific TRE is preferentially functional in a limited population (or type) of cells, e.g., prostate cells, liver cells, melanoma cells, etc. Accordingly, in some embodiments, the TRE used is preferentially functional in any of the following tissue types: prostate; liver; breast; urothelial (bladder); colon; lung; ovarian; pancreas; stomach; and uterine.

As is readily appreciated by one skilled in the art, a TRE is a polynucleotide sequence, and, as such, can exhibit function over a variety of sequence permutations. Methods of nucleotide substitution, addition, and deletion are known in the art, and readily-available functional assays (such as the CAT or luciferase reporter gene assay) allow one of ordinary skill to determine whether a sequence variant exhibits requisite cell-specific transcription regulatory function. Hence, functionally preserved variants of TREs, comprising nucleic acid substitutions, additions, and/or deletions, can be used in the vectors disclosed herein. Accordingly, variant TREs retain function in the target cell but need not exhibit maximal function. In fact, maximal transcriptional activation activity of a TRE may not always be necessary to achieve a desired result, and the level of induction afforded by a fragment of a TRE may be sufficient for certain applications. For example, if used for treatment or palliation of a disease state, less-than-maximal responsiveness may be sufficient if, for example, the target cells are not especially virulent and/or the extent of disease is relatively confined.

Certain base modifications may result in enhanced expression levels and/or cell-specificity. For example, nucleic acid sequence deletions or additions within a TRE can move transcription regulatory protein binding sites closer or farther away from each other than they exist in their normal configuration, or rotate them so they are on opposite sides of the DNA helix, thereby altering spatial relationship among TRE-bound transcription factors, resulting in a decrease or increase in transcription, as is known in the art. Thus, while not wishing to be bound by theory, the present disclosure contemplates the possibility that certain modifications of a TRE will result in modulated expression levels as directed by the TRE, including enhanced cell-specificity. Achievement of enhanced expression levels may be especially desirable in the case of more aggressive forms of neoplastic growth, and/or when a more rapid and/or aggressive pattern of cell killing is warranted (for example, in an immunocompromised subject).

A TRE for use in the present vectors may or may not comprise a silencer. The presence of a silencer (i.e., a negative regulatory element known in the art) can assist in shutting off transcription (and thus replication) in non-target cells. Thus, presence of a silencer can confer enhanced cell-specific vector replication by more effectively preventing replication in non-target cells. Alternatively, lack of a silencer may stimulate replication in target cells, thus conferring enhanced target cell-specificity.

Transcriptional activity directed by a TRE (including both inhibition and enhancement) can be measured in a number of ways known in the art (and described in more detail below), but is generally measured by detection and/or quantitation of mRNA and/or of a protein product encoded by the sequence under control of (i.e., operably linked to) a TRE.

As discussed herein, a TRE can be of varying lengths, and of varying sequence composition. The size of a heterologous TRE will be determined in part by the capacity of the viral vector, which in turn depends upon the contemplated form of the vector. Generally minimal sizes are preferred for TREs, as this provides potential room for insertion of other sequences which may be desirable, such as transgenes and/or additional regulatory sequences. In a preferred embodiment, such an additional regulatory sequence is a self-processing cleavage sequences such as a 2A or 2A-like sequence.

By way of example, an adenoviral vector can be packaged with extra sequences totaling up to about 105% of the genome size, or approximately 1.8 kb, without requiring deletion of viral sequences. If non-essential sequences are removed from the adenovirus genome, an additional 4.6 kb of insert can be tolerated (i.e., for a total insertion capacity of about 6.4 kb).

In the case of adenoviral vectors, in order to minimize non-specific replication, endogenous (adenovirus) TREs (i.e., the native E1A and/or E1B promoter) are preferably removed from the vector. Besides facilitating target cell-specific replication, removal of endogenous TREs also provides greater insert capacity in a vector, which is of special concern if an adenoviral vector is to be packaged within a virus particle. Even more importantly, deletion of endogenous TREs prevents the possibility of a recombination event whereby a heterologous TRE is deleted and the endogenous TRE assumes transcriptional control of its respective adenovirus coding sequences (thus allowing non-specific replication). In one embodiment, an adenoviral vector is constructed such that the endogenous transcription control sequences of one or more adenoviral genes are deleted and replaced by one or more heterologous TREs. However, endogenous TREs can be maintained in the adenovirus vector(s), provided that sufficient cell-specific replication preference is preserved. These embodiments are constructed by inserting heterologous TREs between an endogenous TRE and a gene coding segment required for replication. Requisite cell-specific replication preference is determined by conducting assays that compare replication of the adenovirus vector in a cell which allows function of the heterologous TREs with replication in a cell which does not.

Generally, a TRE will increase replication of a vector in a target cell by at least about 2-fold, preferably at least about 5-fold, preferably at least about 10-fold more preferably at least about 20-fold, more preferably at least about 50-fold, more preferably at least about 100-fold, more preferably at least about 200-fold, even more preferably at least about 400- to about 500-fold, even more preferably at least about 1000-fold, compared to basal levels of replication in the absence of a TRE. The acceptable differential can be determined empirically (by measurement of mRNA levels using, for example, RNA blot assays, RNase protection assays or other assays known in the art) and will depend upon the anticipated use of the vector and/or the desired result.

Replication-competent viral vectors directed at specific target cells can be generated using TREs that are preferentially functional in a target cell. In one embodiment of the present invention, a target cell-specific or cell status-specific, heterologous TRE is tumor cell-specific. A vector can comprise a single tumor cell-specific TRE or multiple heterologous TREs which are tumor cell-specific and functional in the same cell. In another embodiment, a vector comprises one or more heterologous TREs which are tumor cell-specific and additionally comprises one or more heterologous TREs Which are tissue specific, whereby all TREs are functional in the same cell.

In a preferred embodiment for the oncolytic adenovirus platform, bicistronic or multicistronic cassettes containing a self processing cleavage sequence such as a 2A or 2A-like sequence comprise adenoviral early viral genes (E1A, E1B, E2, E3, and/and or E4) or genes expressed later in the viral life cycle (fiber, penton, and hexon).

In certain instances, it may be desirable to enhance the degree and/or rate of cytotoxic activity, due to, for example, the relatively refractory nature or particular aggressiveness of the cancerous target cell. An example of a viral gene that contributes to cytotoxicity includes, but is not limited to, the adenovirus death protein (ADP) gene. In another embodiment disclosed herein, the adenovirus comprises the adenovirus E1B gene which has a deletion in or of its endogenous promoter. In other embodiments disclosed herein, the 19-kDa region of E1B is deleted.

To provide enhanced cytotoxicity to target cells, one or more transgenes having a cytotoxic effect may be present in the vector. Additionally, or alternatively, an adenovirus gene that contributes to cytotoxicity and/or cell death, such as the adenovirus death protein (ADP) gene, can be included in the vector, optionally under the selective transcriptional control of a heterologous TRE and optionally under the translational control of a self-processing cleavage sequence, such as a 2A or 2A-like sequence. This could be accomplished by coupling the target cell-specific cytotoxic activity with cell-specific expression of, a heterologous gene or transgene, for example, HSV-tk and/or cytosine deaminase (cd) and/or nitroreductase. Cancer cells can be induced to be conditionally sensitive to the antiviral drug ganciclovir after transduction with HSV-tk. Ganciclovir is converted by HSV-tk into its triphosphate form by cellular enzymes and incorporated into the DNA of replicating mammalian cells leading to inhibition of DNA replication and cell death. Cytosine deaminase renders cells capable of metabolizing 5-fluorocytosine (5-FC) to the chemotherapeutic agent 5-fluorouracil (5-FU). Other desirable transgenes that may be introduced via an adenovirus vector(s) include genes encoding cytotoxic proteins, such as the A chains of diphtheria toxin, ricin or abrin (Palmiter et al. (1987) Cell 50: 435; Maxwell et al. (1987) Mol. Cell. Biol. 7: 1576; Behringer et al. (1988) Genes Dev. 2: 453; Messing et al. (1992) Neuron 8: 507; Piatak et al. (1988) J. Biol. Chem. 263: 4937; Lamb et al. (1985) Eur. J. Biochem. 148: 265; Frankel et al. (1989) Mol. Cell. Biol. 9: 415); genes encoding a factor capable of initiating apoptosis (e.g., Fas, Bax, Caspase, TRAIL, Fas ligands, and the like); tumor suppressor gene such as p53, RB, p16, p17, W9 and the like; sequences encoding antisense transcripts or ribozymes, which among other capabilities may be directed to mRNAs encoding proteins essential for proliferation, such as structural proteins, or transcription factors; viral or other pathogenic proteins, where the pathogen proliferates intracellularly; genes that encode an engineered cytoplasmic variant of a nuclease (e.g. RNase A) or protease (e.g. trypsin, papain, proteinase K, carboxypeptidase, etc.), an anti-angiogenic gene such as endostatin, angiostatin, sVEGFR3, VEGF-TRAP or a fusogenic gene such as the GaLV envelope protein, V22, VSV and the like.

Any of a number of heterologous therapeutic genes or transgenes may be included in the replication competent viral vectors of the invention including, but not limited to cytokines, antigens, transmembrane proteins, and the like, such as IL-1, -2, -6, -12, GM-CSF, G-CSF, M-CSF, IFN-α, -β, -χ, TNF-α, -β, TGF-α, -β, NGF, MDA-7 (Melanoma differentiation associated gene-7, mda-7/interleukin-24), and the like.

Typically, the aforementioned bicistronic or multicistronic cassettes are placed under the control of a transcriptional response element, generally a cell type or cell status associated transcriptional regulatory element that is preferentially expressed in cancer or tumor cells. Accordingly, the therapeutic gene included in a given construct will vary dependent upon the type of cancer under treatment.

As is known in the art, activity of TREs can be inducible. Inducible TREs generally exhibit low activity in the absence of inducer, and are up-regulated in the presence of an inducer. Inducers include, for example, nucleic acids, polypeptides, small molecules, organic compounds and/or environmental conditions such as temperature, pressure or hypoxia. Inducible TREs may be preferred when expression is desired only at certain times or at certain locations, or when it is desirable to titrate the level of expression using an inducing agent. For example, transcriptional activity from the PSE-TRE, PB-TRE and hKLK2-TRE is inducible by androgen, as described herein and in PCT/US98/04080, expressly incorporated by reference herein. Accordingly, in one embodiment of the present invention, the adenovirus vector comprises an inducible heterologous TRE.

A TRE as used in the present invention can be present in a variety of configurations. A TRE can comprise multimers. For example, a TRE can comprise a tandem series of at least two, at least three, at least four, or at least five target cell-specific TREs. These multimers may also contain heterologous promoter and/or enhancer sequences. Alternatively, a TRE can comprise one or more promoter regions along with one or more enhancer regions. TRE multimers can also comprise promoter and/or enhancer sequences from different genes. The promoter and enhancer components of a TRE can be in any orientation with respect to each other and can be in any orientation and/or any distance from the coding sequence of interest, as long as the desired cell-specific transcriptional activity is obtained.

As used herein, a TRE derived from a specific gene is referred to by the gene from which it was derived and is a polynucleotide sequence which regulates transcription of an operably linked polynucleotide sequence in a host cell that expresses the gene. For example, as used herein, a “human glandular kallikrein transcriptional regulatory element”, or “hKLK2-TRE” is a polynucleotide sequence, preferably a DNA sequence, which increases transcription of an operably linked polynucleotide sequence in a host cell that allows an hKLK2-TRE to function, such as a cell (preferably a mammalian cell, even more preferably a human cell) that expresses androgen receptor, such as a prostate cell. An hKLK2-TRE is thus responsive to the binding of androgen receptor and comprises at least a portion of an hKLK2 promoter and/or an hKLK2 enhancer (i.e., the ARE or androgen receptor binding site). A human glandular kallikrein enhancer and adenoviral vectors comprising the enhancer are described in WO99/06576, expressly incorporated by reference herein.

As used herein, a “probasin (PB) transcriptional regulatory element”, or “PB-TRE” is a polynucleotide sequence, preferably a DNA sequence, which selectively increases transcription of an operably-linked polynucleotide sequence in a host cell that allows a PB-TRE to function, such as a cell (preferably a mammalian cell, more preferably a human cell, even more preferably a prostate cell) that expresses androgen receptor. A PB-TRE is thus responsive to the binding of androgen receptor and comprises at least a portion of a PB promoter and/or a PB enhancer (i.e., the ARE or androgen receptor binding site). Adenovirus vectors specific for cells expressing androgen are described in WO98/39466, expressly incorporated by reference herein.

As used herein, a “prostate-specific antigen (PSA) transcriptional regulatory element”, or “PSA-TRE”, or “PSE-TRE” is a polynucleotide sequence, preferably a DNA sequence, which selectively increases transcription of an operably linked polynucleotide sequence in a host cell that allows a PSA-TRE to function, such as a cell (preferably a mammalian cell, more preferably a human cell, even more preferably a prostate cell) that expresses androgen receptor. A PSA-TRE is thus responsive to the binding of androgen receptor and comprises at least a portion of a PSA promoter and/or a PSA enhancer (i.e., the ARE or androgen receptor binding site). A tissue-specific enhancer active in prostate and use in adenoviral vectors is described in WO95/19434 and WO97/01358, each of which is expressly incorporated by reference herein.

As used herein, a “carcinoembryonic antigen (CEA) transcriptional regulatory element”, or “CEA-TRE” is a polynucleotide sequence, preferably a DNA sequence, which selectively increases transcription of an operably linked polynucleotide sequence in a host cell that allows a CEA-TRE to function, such as a cell (preferably a mammalian cell, even more preferably a human cell) that expresses CEA. The CEA-TRE is responsive to transcription factors and/or co-factor(s) associated with CEA-producing cells and comprises at least a portion of the CEA promoter and/or enhancer. Adenovirus vectors specific for cells expressing carcinoembryonic antigen are described in WO98/39467, expressly incorporated by reference herein.

As used herein, an “alpha-fetoprotein (AFP) transcriptional regulatory element”, or “AFP-TRE” is a polynucleotide sequence, preferably a DNA sequence, which selectively increases transcription (of an operably linked polynucleotide sequence) in a host cell that allows an AFP-TRE to function, such as a cell (preferably a mammalian cell, even more preferably a human cell) that expresses AFP. The AFP-TRE is responsive to transcription factors and/or co-factor(s) associated with AFP-producing cells and comprises at least a portion of the AFP promoter and/or enhancer. Adenovirus vectors specific for cells expressing alpha fetoprotein are described in WO98/39465, expressly incorporated by reference herein.

As used herein, an “a mucin gene (MUC) transcriptional regulatory element”, or “MUC1-TRE” is a polynucleotide sequence, preferably a DNA sequence, which selectively increases transcription (of an operably-linked polynucleotide sequence) in a host cell that allows a MUC 1-TRE to function, such as a cell (preferably a mammalian cell, even more preferably a human cell) that expresses MUC1. The MUC1-TRE is responsive to transcription factors and/or co-factor(s) associated with MUC1-producing cells and comprises at least a portion of the MUC1 promoter and/or enhancer.

As used herein, a “urothelial cell-specific transcriptional response element”, or “urothelial cell-specific TRE” is polynucleotide sequence, preferably a DNA sequence, which increases transcription of an operably linked polynucleotide sequence in a host cell that allows a urothelial-specific TRE to function, i.e., a target cell. A variety of urothelial cell-specific TREs are known, are responsive to cellular proteins (transcription factors and/or co-factor(s)) associated with urothelial cells, and comprise at least a portion of a urothelial-specific promoter and/or a urothelial-specific enhancer. Exemplary urothelial cell specific transcriptional regulatory sequences include a human or rodent uroplakin (UP), e.g., UPI, UPII, UPIII and the like. Human urothelial cell specific uroplakin transcriptional regulatory sequences and adenoviral vectors comprising the same are described in WO01/72994, expressly incorporated by reference herein.

As used herein, a “melanocyte cell-specific transcriptional response element”, or “melanocyte cell-specific TRE” is a polynucleotide sequence, preferably a DNA sequence, which increases transcription of an operably linked polynucleotide sequence in a host cell that allows a melanocyte-specific TRE to function, i.e., a target cell. A variety of melanocyte cell-specific TREs are known, are responsive to cellular proteins (transcription factors and/or co-factor(s)) associated with melanocyte cells, and comprise at least a portion of a melanocyte-specific promoter and/or a melanocyte-specific enhancer. Methods are described herein for measuring the activity of a melanocyte cell-specific TRE and thus for determining whether a given cell allows a melanocyte cell-specific TRE to function. Examples of a melanocyte-specific TRE for use in practicing the invention include but are not limited to a TRE derived from the 5′ flanking region of a tyrosinase gene, a tyrosinase related protein-1 gene, a TRE derived from the 5′-flanking region of a tyrosinase related protein-2 gene, a TRE derived from the 5′ flanking region of a MART-1 gene or a TRE derived from the 5′-flanking region of a gene which is aberrantly expressed in melanoma.

In another aspect, the invention provides replication-competent adenoviral vectors comprising a metastatic colon cancer specific TRE derived from a PRL-3 gene operably linked to a gene essential for adenovirus replication. As used herein, a “metastatic colon cancer specific TRE derived from a PRL-3 gene” or a “PRL-3 TRE” is a polynucleotide sequence, preferably a DNA sequence, which selectively increases transcription of an operably linked polynucleotide sequence in a host cell that allows a PRL-3 TRE to function, such as a cell (preferably a mammalian cell, more preferably a human cell, even more preferably a metastatic colon cancer cell). The metastatic colon cancer-specific TRE may comprise one or more regulatory sequences, e.g. enhancers, promoters, transcription factor binding sites and the like, which may be derived from the same or different genes. In one preferred aspect, the PRL-3 TRE comprises a PRL-3 promoter. One preferred PRL-3 TRE is derived from the 0.6 kb sequence upstream of the translational start codon for the PRL-3 gene, described in WO 04/009790, expressly incorporated by reference herein.

In another aspect, the invention provides replication-competent adenoviral vectors comprising a liver cancer specific TREs derived from the CRG-L2 gene operably linked to a gene essential for adenovirus replication. As used herein, a “liver cancer specific TREs derived from the CRG-L2 gene” or a “CRG-L2 TRE” is a polynucleotide sequence, preferably a DNA sequence, which selectively increases transcription of an operably linked polynucleotide sequence in a host cell that allows a CRG-L2 to function, such as a cell (preferably a mammalian cell, more preferably a human cell, even more preferably a hepatocellular carcinoma cell). The hepatocellular carcinoma specific TRE may comprise one or more regulatory sequences, e.g. enhancers, promoters, transcription factor binding sites and the like, which may be derived from the same or different genes. In one preferred aspect, the CRG-L2 TRE may be derived from the 0.8 kb sequence upstream of the translational start codon for the CRG-L2 gene, or from a 0.7 kb sequence contained within the 0.8 kb sequence (residues 119-803); or from an EcoRI to NcoI fragment derived from the 0.8 kb sequence, as described in U.S. Provisional Application Ser. No. 60/511,812, expressly incorporated by reference herein.

In another aspect, the invention provides replication-competent adenoviral vectors comprising an EBV-specific transcriptional regulatory element (TRE) operably linked to a gene essential for adenovirus replication. In one aspect, the EBV specific TRE is derived from a sequence upstream of the translational start codon for the LMP1, LMP2A or LMP2B genes, as further described in U.S. Provisional Application Ser. No. 60/423,203, expressly incorporated by reference herein. The EBV-specific TRE may comprise one or more regulatory sequences, e.g. enhancers, promoters, transcription factor binding sites and the like, which may be derived from the same or different genes.

In yet another aspect, the invention provides replication-competent adenoviral vectors comprising a hypoxia-responsive element (“HRE”) operably linked to a gene essential for adenovirus replication. HRE is a transcriptional regulatory element comprising a binding site for the transcriptional complex HIF-1, or hypoxia inducible factor-1, which interacts with a in the regulatory regions of several genes, including vascular endothelial growth factor, and several genes encoding glycolytic enzymes, including enolase-1. Accordingly, in one embodiment, an adenovirus vector comprises an adenovirus gene, preferably an adenoviral gene essential for replication, under transcriptional control of a cell status-specific TRE such as a HRE, as further described in WO 00/15820, expressly incorporated by reference herein.

Internal Ribosome Entry Site (IRES)

To express two or more proteins from a single viral or non-viral vector, an internal ribosome entry site (IRES) sequence is commonly used to drive expression of the second, third, fourth gene, etc. Although the use of an IRES is considered to be the state of the art by many, when two genes are linked via an IRES, the expression level of the second gene is often significantly reduced (Furler et al., Gene Therapy 8:864-873 (2001)). In fact, the use of an IRES to control transcription of two or more genes operably linked to the same promoter can result in lower level expression of the second, third, etc. gene relative to the gene adjacent the promoter. In addition, an IRES sequence may be sufficiently long to present issues with the packaging limit of the vector, e.g., the ECMV IRES has a length of 507 base pairs.

IRES elements were first discovered in picornavirus mRNAs (Jackson R J, Howell M T, Kaminski A (1990) Trends Biochem Sci 15(12):477-83) and Jackson R J and Kaminski, A. (1995) RNA 1(10):985-1000). Examples of IRESs generally employed by those of skill in the art include those referenced in Table I, as well as those described in U.S. Pat. No. 6,692,736. Examples of IRESs known in the art include, but are not limited to IRESs obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. (1998) Mol. Cell. Biol. 18(11):6178-6190), the fibroblast growth factor 2 (FGF-2), and insulin-like growth factor (IGFH), the translational initiation factor eIF4G and yeast transcription factors TFIID and HAP4, and the encephelomycarditis virus (EMCV) which is commercially available from Novagen (Duke et al. (1992) J. Virol 66(3):1602-9). IRESs have also been reported in different viruses such as cardiovirus, rhinovirus, aphthovirus, HCV, Friend murine leukemia virus (FrMLV) and Moloney murine leukemia virus (MoMLV). As used herein, the term “IRES” encompasses functional variations of IRES sequences as long as the variation is able to promote direct internal ribosome entry to the initiation codon of a cistron. An IRES may be mammalian, viral or protozoan.

TABLE 1
LITERATURE REFERENCES FOR IRES

| IRES Host | Example | Reference |
|---|---|---|
| Picornavirus | HAV | Glass et al., 1993. Virol 193: |
| | | 842-852 |
| | EMCV | Jang & Wimmer, 1990. Gene Dev 4: |
| | | 1560-1572 |
| | Poliovirus | Borman et al., 1994. EMBO J 13: |
| | | 3149-3157 |
| HCV and | HCV | Tsukiyama-Kohara et al., 1992. |
| pestivirus | | J Virol 66: 1476-1483 |
| | BVDV | Frolov I et al., 1998. RNA. 4: |
| | | 1418-1435 |
| Leishmania | LRV-1 | Maga et al., 1995. Mol Cell Biol |
| virus | | 15: 4884-4889 |
| Retroviruses | MoMLV | Torrent et al., 1996. Hum Gene |
| | VL30 (Harvey | Ther 7: 603-612 |
| | murine | |
| | sarcoma virus) | |
| | REV | Lopez-Lastra et al., 1997. Hum Gene |
| | | Ther 8: 1855-1865 |
| Eukaryotic | BiP | Macejak & Sarnow, 1991. Nature 353: |
| mRNA | | 90-94 |
| | antennapedia | Oh et al., 1992. Gene & Dev 6: |
| | mRNA | 1643-1653 |
| | FGF-2 | Vagner et al., 1995. Mol Cell Biol 15: |
| | | 35-44 |
| | PDGF-B | Bernstein et al., 1997. J Biol Chem 272: |
| | | 9356-9362 |
| | IGFII | Teerink et al., 1995. Biochim Biophys |
| | | Acta 1264: 403-408 |
| | eIF4G | Gan & Rhoads, 1996. J Biol Chem 271: |
| | | 623-626 |
| | VEGF | Stein et al., 1998. Mol Cell Biol 18: |
| | | 3112-3119; Huez et al., 1998. |
| | | Mol Cell Biol 18: 6178-6190 |

The IRES promotes direct internal ribosome entry to the initiation codon of a downstream cistron, leading to cap-independent translation. Thus, the product of a downstream cistron can be expressed from a bicistronic (or multicistronic) mRNA, without requiring either cleavage of a polyprotein or generation of a monocistronic mRNA. Commonly used internal ribosome entry sites are approximately 450 nucleotides in length and are characterized by moderate conservation of primary sequence and strong conservation of secondary structure. The most significant primary sequence feature of the IRES is a pyrimidine-rich site whose start is located approximately 25 nucleotides upstream of the 3′ end of the IRES. See Jackson et al., 1990.

Three major classes of picornavirus IRES have been identified and characterized: (1) the cardio- and aphthovirus class (for example, the encephelomycarditis virus, Jang et al. (1990) Gene Dev 4:1560-1572); (2) the entero- and rhinovirus class (for example, polioviruses, Borman et al. (1994) EMBO J. 13:314903157); and (3) the hepatitis A virus (HAV) class, Glass et al. (1993) Virol 193:842-852). For the first two classes, two general principles apply. First, most of the 450-nucleotide sequence of the IRES functions to maintain particular secondary and tertiary structures conducive to ribosome binding and translational initiation. Second, the ribosome entry site is an AUG triplet located at the 3′ end of the IRES, approximately 25 nucleotides downstream of a conserved oligopyrimidine tract. Translation initiation can occur either at the ribosome entry site (cardioviruses) or at the next downstream AUG (entero/rhinovirus class). Initiation occurs at both sites inaphthoviruses.

HCV and pestiviruses such as bovine viral diarrhea virus (BVDV) or classical swine fever virus (CSFV) have 341 nt and 370 nt long 5′-UTR respectively. These 5′-UTR fragments form similar RNA secondary structures and can have moderately efficient IRES function (Tsukiyama-Kohara et al. (1992) J. Virol. 66:1476-1483; Frolov I et al., (1998) RNA 4:1418-1435). Recent studies showed that both Friend-murine leukemia virus (MLV) 5′-UTR and rat retrotransposon virus-like 30S (VL30) sequences contain IRES structure of retroviral origin (Torrent et al. (1996) Hum Gene Ther 7:603-612).

In eukaryotic cells, translation is normally initiated by the ribosome scanning from the capped mRNA 5′ end, under the control of initiation factors. However, several cellular mRNAs have been found to have IRES structure to mediate the cap-independent translation (van der Velde, et al. (1999) Int J Biochem Cell Biol. 31:87-106). Examples are immunoglobulin heavy-chain binding protein (BiP) (Macejak et al. (1991) Nature 353:90-94), antennapedia mRNA of Drosophilan (Oh et al. (1992) Gene and Dev 6:1643-1653), fibroblast growth factor-2 (FGF-2) (Vagner et al. (1995) Mol Cell Biol 15:35-44), platelet-derived growth factor B (PDGF-B) (Bernstein et al. (1997) J Biol Chem 272:9356-9362), insulin-like growth factor II (Teerink et al. (1995) Biochim Biophys Acta 1264:403-408), and the translation initiation factor eIF4G (Gan et al. (1996) J Biol Chem 271:623-626). Recently, vascular endothelial growth factor (VEGF) was also found to have IRES element (Stein et al. (1998) Mol Cell Biol 18:3112-3119; Huez et al. (1998) Mol Cell Biol 18:6178-6190).

An IRES sequence may be tested and compared to a 2A sequence as shown in Example 2. In one exemplary protocol a test vector or plasmid is generated with one transgene, such as PF-4 or VEGF-TRAP, placed under translational control of an IRES, 2A or 2A-like sequence to be tested. A cell is transfected with the vector or plasmid containing the IRES- or 2A-reporter gene sequences and an assay is performed to detect the presence of the transgene. In one illustrative example, the test plasmid comprises co-transcribed PF-4 and VEGF-TRAP coding sequences transcriptionally driven by a CMV promoter wherein the PF-4 or VEGF-TRAP coding sequence is translationally driven by the IRES, 2A or 2A-like sequence to be tested. Host cells are transiently transfected with the test vector or plasmid by means known to those of skill in the art and assayed for the expression of the transgene.

An IRES may be prepared using standard recombinant and synthetic methods known in the art. For cloning convenience, restriction sites may be engineered into the ends of the IRES fragments to be used.

Internal ribosome entry sites have also proven to be a popular mechanism for controlling the expression of transgenes in replication-competent or -defective vector systems [Tahara H, et al., J. Immunol. 1995 Jun. 15;154(12):6466-74; Urabe M. et al., Gene. 1997 Oct. 24;200(1-2):157-62; and Zhou Y. et al., Hum Gene Ther. 1998 Feb. 10;9(3):287-93. The utility of internal ribosome entry sites is limited by poor expression of the downstream cistrons in multicistronic cassettes (Zhou Y. et al., Hum Gene Ther. 1998 and Mizuguchi H. et al., Mol Ther. 2000 April;1(4):376-82). In the case of oncolytic adenoviruses, an IRES (the smallest known sequence of which is 230 base pairs) can consume valuable room in a space-limited genome (Urabe M. et al., Gene. 1997 Oct. 24;200(1-2):157-62).

Self-Processing Cleavage Sites or Sequences

A “self-processing cleavage site” or “self-processing cleavage sequence” as defined above refers to a DNA or amino acid sequence, wherein upon translation, rapid intramolecular (cis) cleavage of a polypeptide comprising the self-processing cleavage site occurs to result in expression of discrete mature protein or polypeptide products. Such a “self-processing cleavage site”, may also be referred to as a post-translational or co-translational processing cleavage site, exemplified herein by a 2A site, sequence or domain. It has been reported that a 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly, 2001). Alternatively, a 2A site, sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

The Foot and Mouth Disease Virus 2A oligopeptide has previously been demonstrated to mediate the translation of two sequential proteins through a ribosomal skip mechanism (Donnelly M L. et al., J Gen Virol. 2001 May;82(Pt 5):1013-25, Szymczak A L. et al., Nat Biotechnol. 2004 May;22(5):589-94., Klump H. et al., Gene Ther. 2001 May;8(10):811-7; De Felipe P. et al., Hum Gene Ther. 2000 Sep. 1; 11(13): 1921-31; Halpin C. et al., Plant J. 1999 February;17(4):453-9; Mattion N M. et al., J. Virol. 1996 November;70(11):8124-7; and de Felipe P. et al., Gene Ther. 1999 February;6(2):198-208). Multiple proteins are encoded as a single open reading frame (ORF). During translation in a bicistronic system, the presence of the FMDV 2A sequence at the 3′ end of the upstream gene abrogates the peptide bond formation with the downstream cistron, resulting in a “ribosomal skip” and the attachment of the translated FMDV 2A oligopeptide to the upstream protein (Donnelly M L. et al., J Gen Virol. 2001 May;82(Pt 5):1013-25). Processing occurs in a stoichiometric fashion, estimated to be as high as 90-99%, resulting in a near molar equivalency of both protein species (Donnelly M L. et al., J Gen Virol. 2001 May;82(Pt 5):1027-41). Furthermore, through deletion analysis the amino acid sequence-dependent processing activity has been localized to a small section at the c-terminal end of the FMDV 2A oligopeptide (Ryan M D. et al., EMBO J. 1994 Feb 15;13(4):928-33). Most members of the Picornavirus family (of which FMDV belongs) use similar mechanisms of cotranslational processing to generate individual proteins (Donnelly M L. et al., J Gen Virol. 2001 May;82(Pt 5):1027-41). In fact, publications have shown that fragments as small as 13 amino acids can cause the ribosomal skip (Ryan M D. et al., EMBO J. 1994 Feb. 15;13(4):928-33). Incorporation of truncated versions of the peptide in bicistronic vector systems has demonstrated that almost all of the processing activity is preserved even in non-viral vector systems (Donnelly M L. et al., J Gen Virol. 2001 May;82(Pt 5):1027-41). Up to four genes have been efficiently expressed under a single promoter by strategic placement of these types of elements (Szymczak A L. et al., Nat Biotechnol. 2004 May;22(5):589-94.). Therefore, self-processing cleavage sites such as the FMDV 2A oligopeptide provide advantages in order to overcome the size and efficiency constraints of an IRES.

Although the mechanism is not part of the invention, the activity of a 2A-like sequence may involve ribosomal skipping between codons which prevents formation of peptide bonds (de Felipe et al., Human Gene Therapy 11: 1921-1931 (2000); Donnelly et al., J. Gen. Virol. 82:1013-1025 (2001)), although it has been considered that the domain acts more like an autolytic enzyme (Ryan et al., Virol. 173:35-45 (1989). Studies in which the Foot and Mouth Disease Virus (FMDV) 2A coding region was cloned into expression vectors and transfected into target cells showed FMDV 2A cleavage of artificial reporter polyproteins in wheat-germ lysate and transgenic tobacco plants (Halpin et al., U.S. Pat. No. 5,846,767; 1998 and Halpin et al., The Plant Journal 17:453-459, 1999); Hs 683 human glioma cell line (de Felipe et al., Gene Therapy 6:198-208, 1999); hereinafter referred to as “de Felipe II”); rabbit reticulocyte lysate and human HTK-143 cells (Ryan et al., EMBO J. 13:928-933 (1994)); and insect cells (Roosien et al., J. Gen. Virol. 71:1703-1711, 1990). The FMDV 2A-mediated cleavage of a heterologous polyprotein has been shown for IL-12 (p40/p35 heterodimer; Chaplin et al., J. Interferon Cytokine Res. 19:235-241, 1999). The reference demonstrates that in transfected COS-7 cells, FMDV 2A mediated the cleavage of a p40-2A-p35 polyprotein into biologically functional subunits p40 and p35 having activities associated with IL-12.

The FMDV 2A sequence has been incorporated into retroviral vectors, alone or combined with different IRES sequences to construct bicistronic, tricistronic and tetracistronic vectors. The efficiency of 2A-mediated gene expression in animals was demonstrated by Furler (2001) using recombinant adeno-associated viral (AAV) vectors encoding a-synuclein and EGFP or Cu/Zn superoxide dismutase (SOD-1) and EGFP linked via the FMDV 2A sequence. EGFP and a-synuclein were expressed at substantially higher levels from vectors which included a 2A sequence relative to corresponding IRES-based vectors, while SOD-1 was expressed at comparable or slightly higher levels. Furler also demonstrated that the 2A sequence results in bicistronic gene expression in vivo after injection of 2A-containing AAV vectors into rat substantia nigra.

For the present invention, the DNA sequence encoding a self-processing cleavage site is exemplified by viral sequences derived from a picomavirus, including but not limited to an entero-, rhino-, cardio-, aphtho- or Foot-and-Mouth Disease Virus (FMDV). In a preferred embodiment, the self-processing cleavage site coding sequence is derived from a FMDV. Self-processing cleavage sites include but are not limited to 2A and 2A-like sites, sequences or domains (Donnelly et al., J. Gen. Virol. 82:1027-1041 (2001).

Positional subcloning of a 2A sequence between two or more heterologous DNA sequences for the inventive vector construct allows the delivery and expression of two or more open reading frames by operable linkage to a single promoter. FMDV 2A is a polyprotein region which functions in the FMDV genome to direct a single cleavage at its own C-terminus, thus functioning in cis. The FMDV 2A domain is typically reported to be about nineteen amino acids in length ((LLNFDLLKLAGDVESNPGP (SEQ ID NO:1); TLNFDLLKLAGDVESNPGP (SEQ ID NO:2); Ryan et al., J. Gen. Virol. 72:2727-2732 (1991)), however oligopeptides of as few as thirteen amino acid residues ((LKLAGDVESNPGP (SEQ ID NO:3)) have also been shown to mediate cleavage at the 2A C-terminus in a fashion similar to its role in the native FMDV polyprotein processing. Alternatively, a vector according to the invention may encode amino acid residues for other 2A-like regions as discussed in Donnelly et al., J. Gen. Virol. 82:1027-1041 (2001) and including but not limited to a 2A-like domain from picornavirus, insect virus, Type C rotavirus, trypanosome repeated sequences or the bacterium, Thermatoga maritima.

Variations of the 2A sequence have been studied for their ability to mediate efficient processing of polyproteins (Donnelly M L L et al. 2001). Exemplary 2A sequences include but are not limited to the sequences presented in Table 2, below:

TABLE 2
TABLE OF EXEMPLARY 2A SEQUENCES

| | |
|---|---|
| LLNFDLLKLAGDVESNPGP | (SEQ ID NO: 1) |
| TLNFDLLKLAGDVESNPGP; | (SEQ ID NO: 2) |
| LKLAGDVESNPGP | (SEQ ID NO: 3) |
| NFDLLKLAGDVESNPGP | (SEQ ID NO: 4) |
| QLLNFDLLKLAGDVESNPGP | (SEQ ID NO: 5) |
| APVKQTLNFDLLKLAGDVESNPGP. | (SEQ ID NO: 6) |
| VTELLYRMKRAETYCPRPLLAIHPTEARHKQKI | (SEQ ID NO: 7) |
| VAPVKQTLNFDLLKLAGDVESNPGP | |
| LLAIHPTEARHKQKIVAPVKQTLNFDLLKLAGD | (SEQ ID NO: 8) |
| VESNPGP | |
| EARHKQKIVAPVKQTLNFDLLKLAGDVESNPGP | (SEQ ID NO: 9) |

Distinct advantages of self-processing cleavage sequences, such as a 2A sequence or a variant thereof are their use in generating vectors expressing self-processing polyproteins. In one exemplary embodiment of the present invention, an adenoviral vector is provided which comprises DNA segments from adenoviral E1A and E1B linked by a self-processing cleavage sequence. In related embodiments, the vector may further comprise the coding sequence for a peptide containing the furin consensus recognition site, R-X-(K/R)-R-.

The small size of the 2A coding sequence makes its use advantageous in vectors with a limited packaging capacity for a coding sequence such as replication competent adenovirus. Elimination of dual promoters reduces promoter interference that may result in reduced and/or impaired levels of expression for each coding sequence.

Distinct advantages of 2A or 2A-like domains are their use in oncolytic (replication competent) viral vectors resulting in a self-processing polyprotein. Any protein/polypeptide/peptide comprising a self-processing polyprotein obtained through the inventive construct are expressed in approximately equimolar amounts following the proteolytic cleavage-like mechanism of the polyprotein in the 2A domain. These proteins may be heterologous to the vector itself, to each other or to the origin of the 2A sequence, thus 2A activity does not discriminate between heterologous proteins and an FMDV-derived polyprotein in its ability to function or mediate cleavage.

The 2A or 2A-like sequence can be incorporated into oncolytic virus constructs as an element in bicistronic or multicistronic cassettes. Such cassettes will incorporate either two or more endogenous viral genes, one or more endogenous viral genes coupled to one or more heterologous coding sequences or transgenes, or two or more coupled transgenes.

The invention contemplates the use of nucleic acid sequence variants that encode a self-processing cleavage site, such as a 2A or 2A-like polypeptide, and nucleic acid coding sequences that have a different codon for one or more of the amino acids relative to that of the parent (native) nucleotide. Such variants are specifically contemplated and encompassed by the present invention. Sequence variants of self-processing cleavage peptides and polypeptides are included within the scope of the invention as well.

As used herein, the term “sequence identity” means nucleic acid or amino acid sequence identity between two or more aligned sequences, when aligned using a sequence alignment program. The terms “% homology” and “% identity” are used interchangeably herein and refer to the level of nucleic acid or amino acid sequence identity between two or more aligned sequences, when aligned using a sequence alignment program. For example, 80% homology means the same thing as 80% sequence identity determined by a defined algorithm under defined conditions.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J. Mol. Biol. 215: 403-410 (1990), with software that is publicly available through the National Center for Biotechnology Information (http://www.ncbi.nlm.nih.gov/), or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981). See, also, Altschul, S. F. et al., 1990 and Altschul, S. F. et al., 1997.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection.

In accordance with the present invention, also encompassed are sequence variants which encode self-processing cleavage polypeptides and polypeptides themselves that have 80, 85, 88, 89, 90, 91, 92, 93, 94, 95, 96, 97, 98, 99% or more sequence identity to the native sequence.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

Moderate and high stringency hybridization conditions are well known in the art (see, for example, Sambrook, et al, 1989, Chapters 9 and 11, and in Ausubel, F. M., et al., 1993. An example of high stringency conditions includes hybridization at about 42° C. in 50% formamide, 5×SSC, 5× Denhardt's solution, 0.5% SDS and 100 mg/ml denatured carrier DNA followed by washing two times in 2×SSC and 0.5% SDS at room temperature and two additional times in 0.1× SSC and 0.5% SDS at 42° C. 2A sequence variants that encode a polypeptide with the same biological activity as the 2A polypeptides described herein and hybridize under moderate to high stringency hybridization conditions are considered to be within the scope of the present invention.

As a result of the degeneracy of the genetic code, a number of coding sequences can be provided which encode the same protein, polypeptide or peptide, such as 2A or a 2A-like peptide. For example, the triplet CGT encodes the amino acid arginine. Arginine is alternatively encoded by CGA, CGC, CGG, AGA, and AGG. Therefore it is appreciated that such substitutions in the coding region fall within the sequence variants that are covered by the present invention.

It is further appreciated that such sequence variants may or may not hybridize to the parent sequence under conditions of high stringency. This would be possible, for example, when the sequence variant includes a different codon for each of the amino acids encoded by the parent nucleotide. Such variants are, nonetheless, specifically contemplated and encompassed by the present invention.

Removal of Self-Processing Peptide Sequences.

One concern associated with the use of self-processing peptides, such as a 2A or 2A-like sequence is that the C terminus of the expressed polypeptide contains amino acids derived from the self-processing peptide, i.e. 2A-derived amino acid residues. These amino acid residues are “foreign” to the host and may elicit an immune response when a protein containing a 2A or 2A-like sequence is expressed in vivo (i.e., in the context of in vivo administration of an oncolytic vector according to the invention).

The invention includes targeted replication-competent viral vectors, e.g., adenoviral vectors, engineered such that an additional proteolytic cleavage site is provided between a first protein or polypeptide coding sequence (the first or 5′ ORF) and the self processing cleavage site as a means for removal of self processing cleavage site derived amino acid residues that are present in the expressed protein product.

Examples of additional proteolytic cleavage sites are furin cleavage sites with the consensus sequence RXK(R)R (SEQ ID NO:10), which can be cleaved by endogenous subtilisin-like proteases, such as furin and other serine proteases. See, e.g., U.S. Ser. No. 10/831,302, expressly incorporated by reference herein, wherein it is demonstrated that self processing 2A amino acid residues at the C terminus of a first expressed protein can be efficiently removed by introducing a furin cleavage site RAKR (SEQ ID NO:11) between the first polypeptide and a self processing 2A sequence. In addition, use of a plasmid containing a 2A sequence and a furin cleavage site adjacent to the 2A sequence was shown to result in a higher level of protein expression than a plasmid containing the 2A sequence alone. This improvement provides a further advantage in that when 2A amino acid residues are removed from the C-terminus of the protein, longer 2A- or 2A like sequences or other self-processing sequences can be used.

In order to avoid the undesired immune responses induced by 2A- or 2A like sequences originating from different species following in vivo administration of a replication-competent viral vector of the invention, the coding sequence for a proteolytic cleavage site may be inserted (using standard methodology known in the art) between the coding sequence for a first protein and the coding sequence for a self-processing peptide so as to remove the self-processing peptide sequence from the resulting viral vector.

Any additional proteolytic cleavage site known in the art that can be expressed using recombinant DNA technology may be employed in practicing the invention. Exemplary additional proteolytic cleavage sites which can be inserted between a polypeptide or protein coding sequence and a self processing cleavage sequence include, but are not limited to a:
a). Furin consensus sequence or site: RXK(R)R (SEQ ID. NO:10);
b). Furin cleavage site RAKR (SEQ ID. NO:11);
c). Factor Xa cleavage sequence or site: IE(D)GR (SEQ ID. NO:12);
d). Signal peptidase I cleavage sequence or site: e.g., LAGFATVAQA (SEQ ID. NO:13); and
e). Thrombin cleavage sequence or site: LVPRGS (SEQ ID. NO:14).
   Preparation of the Viral Vectors of the Invention

The viral vectors of the invention can be prepared using recombinant techniques that are standard in the art. Generally, a target cell-specific TRE is inserted 5′ to the viral gene of interest, preferably a viral replication gene, and in the case of adenovirus one or more early replication genes (although late gene(s) can be used). A target cell-specific TRE can be prepared using oligonucleotide synthesis (if the sequence is known) or recombinant methods (such as PCR and/or restriction enzymes). Convenient restriction sites, either in the natural viral sequence or introduced by methods such as PCR or site-directed mutagenesis, provide an insertion site for a target cell-specific TRE. Accordingly, convenient restriction sites for annealing (i.e., inserting) a target cell-specific TRE can be engineered onto the 5′ and 3′ ends of a UP-TRE using standard recombinant methods, such as PCR.

Polynucleotides used for making viral vectors of the invention may be obtained using standard methods in the art, such as chemical synthesis, recombinant methods and/or obtained from biological sources.

Adenoviral vectors containing all replication-essential elements, with the desired elements (e.g., E1A) under control of a target cell-specific TRE, are conveniently prepared by homologous recombination or in vitro ligation of two plasmids, one providing the left-hand portion of adenovirus and the other plasmid providing the right-hand region, one or more of which contains at least one adenovirus gene under control of a target cell-specific TRE. If homologous recombination is used, the two plasmids should share at least about 500 bp of sequence overlap. Each plasmid, as desired, may be independently manipulated, followed by cotransfection in a competent host, providing complementing genes as appropriate, or the appropriate transcription factors for initiation of transcription from a target cell-specific TRE for propagation of the adenovirus. Plasmids are generally introduced into a suitable host cell such as 293 cells using appropriate means of transduction, such as cationic liposomes. Alternatively, in vitro ligation of the right and left-hand portions of the adenovirus genome can also be used to construct recombinant adenovirus derivative containing all the replication-essential portions of the adenovirus genome. Berkner et al. (1983) Nucleic Acid Research 11: 6003-6020; Bridge et al. (1989) J. Virol. 63: 631-638.

Replication Competent Viral Vectors of the Invention

The adenovirus vectors of the invention comprise target cell specific TREs which direct preferential expression of an operatively linked gene (or genes) in a particular target cell. Examples of target cells include neoplastic cells, although any cell for which it is desirable and/or tolerable to sustain a cytotoxic activity can be a target cell. By combining a viral vector comprising a target cell-specific TRE with a mixture of target and non-target cells, in vitro or in vivo, the vector preferentially replicates in the target cells, causing cytotoxic and/or cytolytic effects. Once the target cells are destroyed due to selective cytotoxic and/or cytolytic activity, replication of the vector(s) is significantly reduced, lessening the probability of runaway infection and undesirable bystander effects. In vitro cultures can be retained to continually monitor the mixture (such as, for example, a biopsy or other appropriate biological sample) for the presence of the undesirable target cell, e.g., a cancer cell in which the target cell-specific TRE is functional. The viral vectors of the present invention can also be used in ex vivo procedures wherein desirable biological samples comprising target cells are removed from a mammal, subjected to exposure to a viral vector of the present invention comprising a target cell-specific TRE and then replaced within the mammal.

The disclosed vectors are designed such that replication is preferentially enhanced in target cells in which the one or more TRE(s) are functional. More than one TRE can be present in a vector, as long as the TREs are functional in the same target cell. However, it is important to note that a given TRE can be functional in more than one type of target cell. For example, the CEA-TRE functions in, among other cell types, gastric cancer cells, colorectal cancer cells, pancreatic cancer cells and lung cancer cells.

The replication competent viral vectors of the present invention comprise an intergenic self-processing cleavage site which links the translation of two or more genes, and therefore represents an improvement over vector constructs which use identical control regions to drive expression of two or more desired genes. The improved vectors of the invention substantially minimize any potential for homologous recombination based on the presence of homologous control regions. Adenoviral vectors with self-processing cleavage sites are described herein in order to exemplify the invention. The viral vectors of the invention provide a number of advantages over the current state of the art including the following (1) use of a self-processing cleavage sites, e.g., a 2A sequence rather than a second TRE or IRES provides additional space in the vector for additional gene(s), such as a therapeutic gene; (2) the 2A sequence can mediate improved expression of the second gene product in bicistronic or multicistronic cassettes relative to that achieved with the current state of the art, e.g., the IRES; and (3) vectors comprising a self-processing cleavage site such as 2A or a 2A-like sequence are stable and in some embodiments provide better specificity than vectors not containing a 2A sequence.

Accordingly, in one aspect of the invention, the viral vectors disclosed herein comprise at least one self-processing cleavage site, e.g., a 2A sequence within a bi- or multi-cistronic transcript, wherein production of the transcript is regulated by a heterologous, target cell-specific TRE or another promoter (such as an endogenous viral promoter or a constitutive promoter capable of high level expression, e.g., the CMV promoter). For viral vectors comprising a second viral gene under control of a 2A sequence, e.g., adenoviral vectors, it is preferred that the endogenous promoter of a gene under translational control of a 2A sequence be deleted so that the endogenous promoter does not interfere with transcription of the second gene. It is also preferred that the second gene be in frame with the 2A sequence.

Introduction of Replication Competent Viral Vectors into Cells

The replication competent viral vectors of the invention find utility in therapeutic methods for the treatment of cancer. The viral vectors of the invention can be used in a variety of forms, including, but not limited to, naked polynucleotide (e.g., DNA) constructs. The viral vectors can, alternatively, comprise polynucleotide constructs that are complexed with agents to facilitate entry into cells, such as cationic liposomes or other cationic compounds such as polylysine; packaged into infectious virus particles (which may render the vector(s) more immunogenic); packaged into other particulate viral forms such as HSV or AAV; complexed with agents (such as PEG) to enhance or dampen an immune response; complexed with agents that facilitate in vivo transfection, such as DOTMA™, DOTAP™, and polyamines.

The viral vector may be delivered to the target cell in a variety of ways, including, but not limited to, liposomes, general transfection methods that are well known in the art, such as calcium phosphate precipitation, electroporation, direct injection, and intravenous infusion. In vitro (ex vivo) techniques include transfection using calcium phosphate, micro-injection into cultured cells (Capecchi, Cell 22:479-488 [1980]), electroporation (Shigekawa et al., BioTechn., 6:742-751 [1988]), liposome-mediated gene transfer (Mannino et al., BioTechn., 6:682-690 [1988]), lipid-mediated gene transfer and the like.

The means of delivery will depend in large part on the particular viral vector (including its form) as well as the type and location of the target cells (i.e., whether the cells are in vitro (ie, ex vivo) or in vivo). If an adenoviral vector comprising an adenovirus polynucleotide is packaged into a whole adenovirus (including the capsid), the adenovirus itself may be employed to further enhance targeting. For example, an adenovirus fiber, shaft or hexon protein may be modified to further increase cell-specificity of cytotoxicity and/or cytolysis.

The delivery route for introducing the recombinant vectors of the present invention in vivo includes, but is not limited to intratumoral, intravenous, intradermal or subcutanaeous injection. In the case of ex vivo gene transfer, the target cells are removed from the host and genetically modified in the laboratory using a vector of the present invention and methods well known in the art, then returned to the subject from which they were derived.

The amount of viral vector to be administered will depend on several factors, such as route of administration, the condition of the subject, the degree of aggressiveness of the disease, the particular target cell-specific TRE employed, and the particular vector construct (i.e., which viral gene or genes is under target cell-specific TRE control) as well as whether the viral vector is used in conjunction with other treatment modalities.

If administered as a packaged virus, typically from about 10⁴ to about 10¹⁴, preferably from about 10⁴ to about 10¹², more preferably from about 10⁴ to about 10¹⁰. If administered as a polynucleotide construct (i.e., not packaged as a virus), about 0.01 μg to about 100 μg can be administered, preferably 0.1 μg to about 500 μg, more preferably about 0.5 μg to about 200 μg. More than one viral vector can be administered, either simultaneously or sequentially. Administrations are typically given periodically, while monitoring any response. Administration can be given, for example, intratumorally, intravenously or intraperitoneally.

If administered as a packaged adenovirus, the vector is administered in an appropriate physiologically acceptable carrier at a dose of about 10⁴ to about 10¹⁴. The multiplicity of infection will generally be in the range of about 0.001 to 100. If administered as a polynucleotide construct (i.e., not packaged as a virus) about 0.01 μg to about 1000 μg of an adenoviral vector can be administered.

A viral vector may be administered one or more times, depending upon the intended use and the immune response potential of the host or may be administered as multiple, simultaneous injections. If an immune response is undesirable, the immune response may be diminished by employing a variety of immunosuppressants, so as to permit repetitive administration, without a strong immune response. If packaged as another viral form, such as HSV, an amount to be administered is based on standard knowledge about that particular virus (which is readily obtainable from, for example, published literature) and can be determined empirically.

Generally, a pharmaceutical composition comprising a viral vector in a pharmaceutically acceptable excipient is administered. Pharmaceutically acceptable excipients are generally known in the art.

The viral vectors of the invention can be used alone or in conjunction with other active agents, such as chemotherapeutics, radiation and/or antibodies that promote the desired objective. Examples of chemotherapeutics which are suitable for suppressing bladder tumor growth are BGC (bacillus Calmett-Guerin); mitomycin-C; cisplatin; thiotepa; doxorubicin; methotrexate; paclitaxel (TAXOL™); ifosfamide; gallium nitrate; gemcitabine; carboplatin; cyclophosphamide; vinblastine; vincristin; fluorouracil; etoposide; bleomycin. Examples of combination therapies include (CISCA (cyclophosphamide, doxorubicin, and cisplatin); CMV (cisplatin, methotrexate, vinblastine); MVMJ (methotrextate, vinblastine, mitoxantrone, carboplain); CAP (cyclophosphamide, doxorubicin, cisplatin); MVAC (methotrexate, vinblastine, doxorubicin, cisplatin). Radiation may also be combined with chemotherapeutic agent(s), for example, radiation with cisplatin. Administration of the chemotherapeutic agents is generally intravesical (directly into the bladder) or intravenous.

Utility of Replication Competent Viral Vectors of the Invention

The subject vectors can be used for a wide variety of purposes, which will vary with the desired or intended result. Accordingly, the present invention includes any of a variety of methods, including but not limited to, therapeutic methods, vaccines, and in the preferred embodiment, cancer therapies. For example, in vivo delivery of a replication competent (recombinant) viral vector may be targeted to a wide variety of organ types including brain, liver, blood vessels, muscle, heart, lung, prostate, skin, a solid tumor, a metastatic tumor, a carcinoma or a rheumatoid joint.

In one embodiment, methods are provided for conferring selective cytotoxicity on cells that allow a target cell-specific TRE to function, preferably cancer cells by contacting the cells with a viral vector described herein. Cytotoxicity can be measured using standard assays in the art, such as dye exclusion, 3H-thymidine incorporation, and/or lysis.

In another embodiment, methods are provided for propagating a viral vector specific for cells which allow a target cell-specific TRE to function, preferably target cancer cells. The list of possible target cells include cells associated with any type of cancer, including, but not limited to colon, breast, cervix, ovarian, stomach, kidneys, bladder, pancreas, head and neck, lymphomas, and leukemias. These methods entail combining a viral vector with the cells, whereby the virus is selectively propagated in the cancer cell. As will be appreciated, the replication competent viral vectors of the invention comprise at least one cell type specific and/or cell status specific regulatory element, in order to facilitate selective replication in target cancer cells.

The invention further provides methods of suppressing tumor cell growth, preferably a tumor cell that allows a target cell-specific TRE to function, comprising contacting a tumor cell with a viral vector of the invention such that the viral vector enters the tumor cell and exhibits selective cytotoxicity for the tumor cell. For these methods, the viral vector may or may not be used in conjunction with other treatment modalities for tumor suppression, such as chemotherapeutic agents, radiation and/or antibodies.

The invention also provides methods of treatment, in which an effective amount of a viral vector described herein is administered to a subject. In vivo administration of a viral vector finds utility in treatment of a subject diagnosed as having cancer and may also find utility in a subject considered to be at risk for developing cancer. Determination of suitability of administering a viral vector of the invention will depend, inter alia, on assessable clinical parameters such as serological indications and histological examination of tissue biopsies.

Another embodiment provides methods for killing cells that allow a target cell-specific TRE to function in a mixture of cells, comprising combining the mixture of cells with a viral vector of the present invention. The mixture of cells is generally a mixture of normal cells and cancerous cells that allow a target cell-specific TRE to function, and can be an in vivo mixture or in vitro mixture.

The invention also includes methods for detecting cells which allow a target cell-specific TRE to function, such as cancer cells, in a biological sample. These methods are particularly useful for monitoring the clinical and/or physiological condition of a subject (i.e., mammal), whether in an experimental or clinical setting. In one method, cells of a biological sample are contacted with a viral vector, and replication of the vector is detected. Alternatively, the sample can be contacted with a viral vector in which a reporter gene is under control of a target cell-specific TRE. When such a viral vector is introduced into a biological sample, expression of the reporter gene indicates the presence of cells that allow a target cell-specific TRE to function. Alternatively, a viral vector can be constructed in which a gene conditionally required for cell survival is placed under control of a target cell-specific TRE. This gene may encode, for example, antibiotic resistance. Later the biological sample is treated with an antibiotic. The presence of surviving cells expressing antibiotic resistance indicates the presence of cells capable of target cell-specific TRE function. A suitable biological sample is one in which cells that allow a target cell-specific TRE to function, such as cancer cells, may be or are suspected to be present. Generally, in mammals, a suitable clinical sample is one in which cancerous cells that allow a target cell-specific TRE to function, such as carcinoma cells, are suspected to be present. Such cells can be obtained, for example, by needle biopsy or other surgical procedure. Cells to be contacted may be treated to promote assay conditions, such as selective enrichment, and/or solubilization. In these methods, cells that allow a target cell-specific TRE to function can be detected using in vitro assays that detect viral proliferation, which are standard in the art. Examples of such standard assays include, but are not limited to, burst assays (which measure virus yield) and plaque assays (which measure infectious particles per cell). Propagation can also be detected by measuring specific viral DNA replication, which are also standard assays known in the art (e.g., PCR, Southern blot and the like).

The invention also provides methods of modifying the genotype of a target cell, comprising contacting the target cell with a viral vector described herein, wherein the viral vector enters the cell.

The invention also provides methods of lowering the levels of a tumor cell marker in a subject, comprising administering to the subject a viral vector of the present invention, wherein the viral vector is selectively cytotoxic toward cells that allow a target cell-specific TRE to function. Tumor serum markers which include but are not limited to PSA and CEA and tumor cell markers such as CK-20 and Her2/neu as well as others may be monitored using immunological assays. For example, enzyme-linked immunosorbent assay (ELISA) of body fluids or immunological staining of cells using antibodies specific for the tumor cell marker are employed. In general, a biological sample is obtained from the subject to be tested, and a suitable assay, such as an ELISA, is performed on the biological sample. For these methods, the adenoviral vector may or may not be used in conjunction with other treatment modalities for tumor suppression, such as chemotherapeutic agents, radiation and/or antibodies.

Compositions and Kits

The present invention also includes compositions, including pharmaceutical compositions, containing the viral vectors described herein. Such compositions are useful for administration in vivo, for example, when measuring the degree of transduction and/or effectiveness of cell killing in a subject. Compositions comprise a viral vector of the invention and a suitable solvent, such as a physiologically acceptable buffer. These are well known in the art. In other embodiments, these compositions comprise a pharmaceutically acceptable excipient. These compositions, which can comprise an effective amount of a viral vector of the invention in a pharmaceutically acceptable excipient, are suitable for systemic or local administration to subjects in unit dosage forms, sterile parenteral solutions or suspensions, sterile non-parenteral solutions or oral solutions or suspensions, oil in water or water in oil emulsions and the like. Formulations for parenteral and nonparenteral drug delivery are known in the art and are set forth in Remington's Pharmaceutical Sciences, 19th Edition, Mack Publishing (1995). Compositions also include lyophilized and/or reconstituted forms of a viral vector (including those packaged as a virus, such as adenovirus) of the invention.

The present invention also encompasses kits containing a viral vector composition of the invention, as described above. These kits can be used for diagnostic and/or monitoring purposes, preferably monitoring. Procedures using these kits can be performed by clinical laboratories, experimental laboratories, medical practitioners, or private subjects. For example, kits embodied by the invention allow for detection of the presence of bladder cancer cells in a suitable biological sample, such as a biopsy specimen.

The kits of the invention comprise a viral vector composition described herein in suitable packaging. The kit may optionally provide additional components that are useful in the procedure, including, but not limited to, buffers, developing reagents, labels, reacting surfaces, means for detection, control samples, instructions, and interpretive information.

The objects of the invention have been achieved by a series of experiments, some of which are described by way of the following non-limiting examples.

### EXPERIMENTAL

Materials and Methods

Cells: 293 cells were obtained from Microbix (Ontario, Canada). A549, Hep3B, Lovo, Panc 1, SW780, WI-38, and MRC-5 cells were obtained from ATCC (Manassas, Va.). HRE cells were obtained from Cambrex (East Rutherford, N.J.). 293, A549, Hep3B, Lovo, Panc 1, SW780, WI-38, and HRE cells are maintained in RPMI 1640 supplemented with 10% fetal bovine serum, 100 units/mL penicillin, 100 micrograms/mL streptomycin, and 2.05 mM L-glutamine. MRC5 cells are maintained in EMEM supplemented with 10% fetal bovine serum, 100 units/mL penicillin, 100 micrograms/mL streptomycin, and 2 mM L-glutamine. All cells are grown at 37 C, 5% CO2.

Viral Amplification, Identification and Titration: Amplified crude viral lysates resulting from co-transfections are passaged on 293 cells and overlayed with solid media to obtain single plaques. Plaques are further passaged two more times on A549 cells and used to infect A549 cells. Following extensive visually observable lysis of the infected A549 cells, crude viral lysates are collected and analyzed by PCR followed by restriction mapping of the PCR amplicons for predicted structural characteristics. Upon confirmation of isolates matching the predicted structural characteristics, crude viral lysates are used to infect larger volumes of A549 cells to generate crude viral lysate stocks. Following titration and structural confirmation, the crude viral lysate stocks are used to infect A549 cells seeded in roller bottles. Upon extensive visually observable lysis, cell pellets are harvested and virus purified by CsCl gradient, dialysis, and finally resuspension in ARCA buffer for storage.

Viral identification: Viral genomic DNA is purified from amplified viral stocks using a Blood Mini Kit (Qiagen; Valencia, Calif.). PCR products are amplified from the purified viral genomic DNA using primers 66.114.2 (5′-gtggcggaacacatgtaagc) and 27.20.2 (5′-aatatcaaatcctcctcgttt), corresponding to positions 133 to 152 and 6148 to 6168, respectively, of the Ad5 genome (Genbank #AY339865). The PCR products are then restriction mapped with the New England Biolabs restriction endonucleases BstX I and Acc I.

Titration: The concentrations of crude viral lysates are determined by standard plaque assay (plaque forming units per mL). Concentrations of purified viruses are measured in both viral particles and plaque forming units. For viral particles, dilution series of lysed virus are measured on a spectrophotometer for DNA mass. Particle number is then determined by the formula outlined by Mittereder et al. [25]:

_{OD}260× dilution factor×(1.1×10¹²)=particles/mL

Virus Yield: Clarified viral lysates are used to infect a panel of cell lines seeded 24 hours earlier at 5×10e5 cells/well in 6-well tissue culture dishes at a multiplicity of infection (MOI) of 2. 4 hours post-transduction, cells are washed twice with Dulbecco's Phosphate Buffered Saline, followed by the return of fresh media. 72 hours post-transduction, cells are scraped into the supernatant and collected. The total lysates are subjected to three freeze/thaw cycles, then serially diluted onto 293 cells seeded 24 hours earlier at 5×10e5 cells/well in 6-well tissue culture dishes. 4 hours post-infection, supernatants are aspirated, and wells overlayed with a solid media containing RPMI, 0.75% low melting point agarose, penicillin/streptomycin, and L-glutamine. 7 days post-infection a second overlay is added to each well. 10 days post-infection plaques are visually scored and averaged to obtain the number of plaque-forming units.

Western Blots: Clarified viral lysates are used to infect A549 cells seeded 24 hours earlier at 5×10e5 cells/well in 6-well tissue culture dishes at an MOI of 10. Twenty-four hours post-infection, cells are scraped into the supernatant, collected, and pelleted. The cell pellets are resuspended in lysis buffer (100 mM NaCl, 20 mM Tris ph 7.5, 10 mM EDTA, 1% deoxycholic acid) supplemented with a Complete, Mini Protease Inhibitor Cocktail (Roche; Indianapolis, Ind.). Protein concentrations of samples are assessed with a protein assay kit (Bio-Rad; Hercules, Calif.). For detection of E1A, 10 micrograms of total protein from each sample are subjected to PAGE (4-12% NuPage Novex Bis-Tris SDS-PAGE) (Invitrogen; Carlsbad, Calif.) in NuPage MOPS running buffer (Invitrogen). Fractions are transferred to an Invitrolon PVDF membrane (Invitrogen) which is probed with either a monoclonal E1A primary antibody (Neomarkers; Fremont, Calif.) or a polyclonal E1A primary antibody (Santa Cruz Biotechnology, Santa Cruz, Calif.) followed by a horseradish peroxidase conjugated secondary antibody. Bound antibody complexes are detected with the Enhanced Chemiluminescence Plus system (Amersham; Buckinghamshire, England). Detection of E1B 55K is performed as above with the following exceptions. The primary antibody is a monoclonal against E1B 55K. (Oncogene Research Products; Boston, Mass.).

Cell Viability Assays: CsCl purified viral stocks are used to infect a panel of cell lines seeded in 96-well tissue culture dishes at 1×10e4 cells/well (Corning). Viruses are serially diluted by a factor of 5, giving a range of MOIs from 1000 to 0.00256. 7 days post-infection, an MTS assay is performed per the manufacturer's instructions (Promega) on the tumor cell lines. The level of absorbance is then assessed as a relative percentage of the conversion of MTS to formazan by uninfected cells (assigned a value of 100% viability, the maximum expected conversion). The above procedure is repeated for the normal cell lines 10 days post-infection.

### Example 1

Construction of Viral Constructs Comprising Self-Processing Cleavage Sequences OV1054 Construct (OV1054).

In one example, a viral vector was constructed by cloning a 2A element in a bicstronic or multicistronic cassette expressing E1A and E1B under the control of the E2F promoter based on the following protocol:

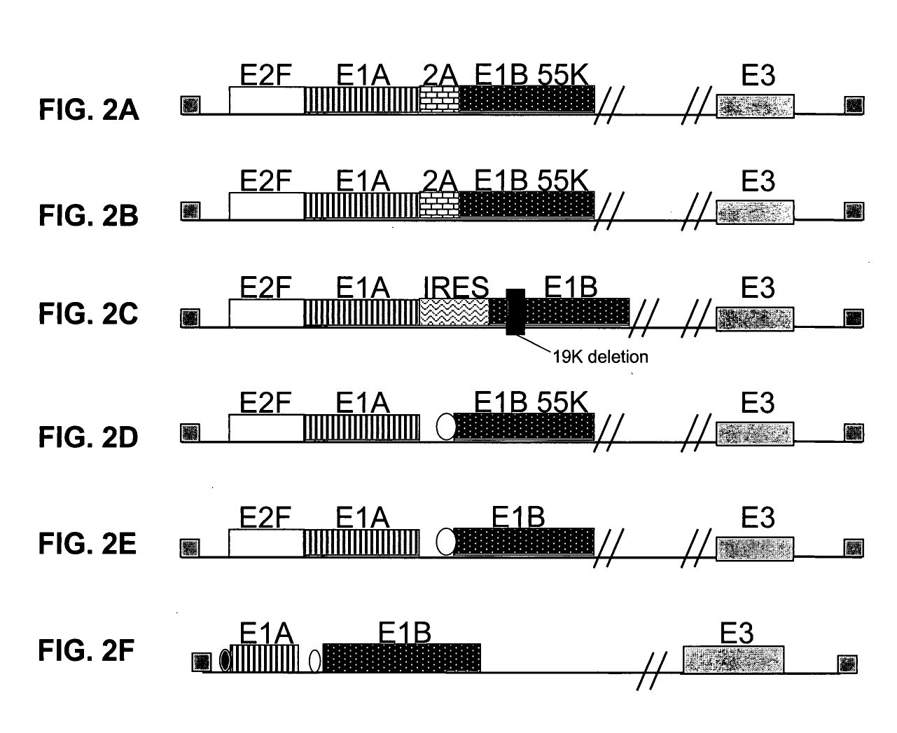

OV1054.11.M.2.2.C (FIG. 2A): The Foot and Mouth Disease Virus 2A (FMDV 2A) oligopeptide was introduced between the E1A and E1B regions of a replication-competent adenovirus vector specific for cells with an Rb-defective pathway. A platform plasmid, CP624 (as described in WO 98/39465 and WO 01/73093), was created by modification of pXC1 (a plasmid containing the wild-type left-hand end of Ad5 from nt 22 to 5790 including the inverted terminal repeat, the packaging sequence, and the E1a and E1b genes in a pBR322 backbone; Microbix). CP624 has a 100 base pair deletion in the E1A/E1B intergenic region with a Sal I site at the junction, deletion of the endogenous E1A promoter with an Age I site upstream of E1A, and a deletion of the E1A polyadenylation signal. The human E2F-1 promoter (SEQ ID NO:15) was amplified from human genomic DNA by PCR using the primers

1405.77.1
(5′-ataccggtggtaccatccggacaaagcctgcgcg) and
1405.77.2
(5′-agaccggtcgagggctcgatcccgctccg).

The human telomerase reverse transcriptase (hTERT) promoter (SEQ ID NO:16) was amplified from pGRN316 (Geron; Menlo Park, Calif.) by PCR with the primers:

1244.39.1:
(5′-aagtcgaccggtaccgtggcggagggactggggac); and
1244.39.2
(5′-aagtcgaccggtgcgggggtggccggggccaggg).

CP624 was modified by placement of the human E2F-1 promoter between the Age I site (such that the human E2F-1 promoter controls E1A expression) and the hTERT promoter between the Sal I site (such that the hTERT promoter controls E1B expression) to yield plasmid CP1498. The C-terminal end of E1A from the Xba I site to the end of the open reading frame was amplified from CP1498 by PCR with the primers:

| | |
|---|---|
| 1460.138.3 | (5′-tgtgtctagagaatgcaatag) and |
| 1460.138.4 | (5′-gatatatgtcgactggcctggggcgtttacagc), |

moving the Sal I site at the 3′ end of the fragment in frame with the E1A coding region. An 84 base pair FMDV 2A segment was PCR amplified from pTREF1ADC101H2ALPRE by primers:

1460.138.1
(5′-atgcagcgtcgacgctccagtaaagcagactcta); and
1460.138.2
(5′-catgatcgtcgactggacctgggttgctctcaac)

placing Sal I sites at both ends. The coding region of E1B 55k from the initiation codon to the Hind III site was amplified from CP1498 by PCR with the primers:

1460.138.5
(5′-gacatgcgtcgacatggagcgaagaaacccatctg); and
1460.138.6
(5′-ccatagaagcttacaccgtgtag)

moving the Sal I site at the 5′ end of the fragment in frame with E1B 55k. The modified E1A, FMDV 2A, and E1B 55k fragments were assembled in plasmid pGEM-3z (Promega; Madison, Wis.) to yield plasmid pGEM-3z.E1A.2A.E1B. The assembled partial cassette of pGEM-3z.E1A.2A.E1B was released by Xba I to Hind III digest and used to replace the region between the same sites of CP1498, resulting in plasmid CP1486. CP1486 contains a multicistronic cassette coding for E1A, FMDV 2A, and E1B 55k in a single, open reading frame under the transcriptional control of the human E2F-1 promoter.

A full-length viral genome was obtained by recombination between CP1486 and plasmid pBHGE3 (a plasmid containing the full wild-type Ad5 genome except for a deletion from nts 188 to 1339) (Microbix). The resulting virus, named OV1054.11.M.2.2.C, features an E1A-FMDV 2A-E1B 55k multicistronic cassette under the transcriptional control of the human E2F—I promoter (FIG. 2A). A two amino acid spacer (-VD-) separates the conserved c-terminal end of FMDV 2A (-NPGP-) from the initiation codon of E1B 55k, resulting in the junction -NPGPVDM-.

OV1057 (FIG. 2B): Construction of OV1057 was similar to OV1054.11.M.2.2.C with the following exceptions. An alternate, 78 base pair version of FMDV 2A was amplified from pTREF1ADC10H2ALPRE by PCR using primers 1460.136.1 and 1460.180.2 (5′-catgatcgggccctggacctgggttgctctcaac) to place Sal I and Apa I ends on the PCR product. An alternate version of E1B 55k from the initiation codon to the Hind III site was amplified from CP1498 by PCR with the primers 1460.180.4 (5′-gatgcatgtcgactagggcccatggagcgaagaaacccatctg) and 1460.138. placing an Apa I site at the 5′ end of the PCR product. These fragments were assembled with the E1A fragment previously used in the construction of OV1054.11.M.2.2.C to generate the plasmid pGEM-3z.E1A.2A(c).E1B. The assembled partial cassette of pGEM-3z.E1A.2A(c).E1B was released by Xba I to Hind III digest and used to replace the region between the same sites of CP1498, resulting in plasmid CP1520. CP1520 contains a multicistronic cassette coding for E1A, FMDV 2A, and E1B 55k in a single, open reading frame under the transcriptional control of the human E2F-1 promoter.

A full-length viral genome was obtained by recombination between CP1520 and plasmid pBHGE3. The resulting virus, named OV1057, features an E1A-FMDV 2A-E 1 B 55k multicistronic cassette under the transcriptional control of the human E2F-1 promoter (FIG. 2B). No amino acid spacer separates the conserved c-terminal end of FMDV 2A (-NPGP-) from the initiation codon of E1B 55k, resulting in the junction -NPGPM-.

OV945 (FIG. 2C): Construction of OV945 was similar to construction of OV1054.11.M.2.2.C with the following exceptions. A modified version of the EMCV IRES from pCITE-3a (Novagen; Madison, Wis.) was amplified by PCR with the primers A (5′-GACGTCGACTAATTCCGGTTATTTTCCA) and B (5′-GACGTCGACATCGTGTTTTTCAAAGGAA) placing Sal I sites at the 5′ and 3′ ends of the PCR product. This modified ECMV IRES was inserted in the Sal I site of CP624 to yield plasmid CP627, the human E2F-1 promoter was inserted in the Age I site, and a Sal I (Klenow) to Bgl II fragment was replaced with a smaller BssH II (Klenow) to Bgl II fragment deleting most of the E1B 19k ORF to yield plasmid CP1493. CP1493 features the native E1A region under the transcriptional control of the human E2F-1 promoter, the ECMV IRES mediating translation of the E1B region, and a large deletion in the E1B 19k coding region.

A full-length viral genome was obtained by recombination of CP 1493 with pBHGE3. The resulting virus, designated OV945, features E1A and E1B 55k under the transcriptional control of the human E2F-1 promoter as well as translation of EIB 55k mediated by the ECMV IRES (FIG. 2C).

OV1056 (FIG. 2D): Construction of OV1056 was similar to construction of OV1054.11.M.2.2.C with the following exceptions. The Xba I to Hind III fragment of pXC 1 was placed in pGEM-3z to generate plasmid pGEM-3z.AB. The E1B 55k coding region from the initiation codon to the Hind III site was amplified from pXC1 by PCR with the primers 1460.180.1 (5′-atgccgtatgcctcatacaggatggagcgaagaaacccatctgagc) and 1460.138.6 to place an EcoN I at the 5′ end of the PCR product. The E1B fragment in pGEM-3z.AB was then replaced via EcoN I to Hind III digest with the E1B 55k PCR product digested with the same enzymes to yield plasmid pGEM-3z.AEB. The Xba I to Hind III fragment of pGEM-3z.AEB was used to replace the equivalent region of CP1498 to yield plasmid CP1488. CP1488 contains the native E1A coding region, the native E1B promoter, and a complete deletion of the portion of the E1B 19k coding region that does not overlap with the E1B 55k coding region. In CP1488, E1B 55k is adjacent to the native promoter with its initiation codon in the position formerly occupied by the same of E1B 19k.

A full-length viral genome was obtained by recombination between CP1488 and plasmid pBHGE3. The resulting virus, named OV1056, features the native E1A region under the transcriptional control of the human E2F-1 promoter, deletion of the non-overlapping portion of the E1B 19k coding region, and E1B 55k under the transcriptional control of the native E1B promoter (FIG. 2D).

OV1054.11.M.1.1.B (FIG. 2E): was isolated from a single plaque following the second passage of OV1054.11.M crude viral lysates through A549 cells. It is believed to have E1A under the transcriptional control of the human E2F-1 promoter and be otherwise identical to wild-type Ad5.

### Example 2

Construction of Oncolytic Virus Comprising 2A and TK

In another example, a viral vector is constructed by cloning a 2A element in a bicistronic cassette expressing E1A and thymidine kinase under the control of the hUPII promoter based on the protocol set forth below:
(a) PCR is used to create and amplify the E1A/2A and 2A/TK junctions using sequence specific primers from the template pTREF1ADC101H2ALPRE to create a PCR product 2A NT;
(b) PCR is used to extend the TK ‘arm’ of 2A INT with sequence specific primers;
(c) PCR is used to extend the E1A ‘arm’ of 2A INT with sequence specific primers;
(d) the two arms are mixed and amplified with sequence specific primers to create the final PCR product E1A-2A-TK which features a 5′ Xba I end and a 3′ Hpa I end;
(e) the PCR product E1A-2A-TK is cut with the restriction enzymes Xba I and Hpa I and cloned into the similarly cut vector CP 1131 to yield CP1512; and
(f) co-transfection of linearized CP1512 (Nru I) with linearized CP1412
(Cla I) and co-transfection of linearized CP 1512 (Nru I) with linearized pBHGE3 (ClaI) by Qiagen Superfect to yield OV1083 and OV1082, respectively.

### Example 3

Testing of Viral Constructs Comprising Self-Processing cleavage Sequences

Virus Design: The effects of the incorporation of the FMDV 2A oligopeptide (in an E1A-E1B multicistronic cassette) on the replication, protein expression, and cytotoxicity of transcriptionally-regulated oncolytic adenoviruses were evaluated. Vectors containing FMDV 2A bicistronic cassette (under the transcriptional control of the human E2F-1 promoter) were compared against similarly transcriptionally-regulated vectors. In each case, the expression of EIB 19k and/or 55k is dependent on a different mechanism. These vectors and their associated controls are described below and shown diagrammatically in FIGS. 2A-F.

OV1054.11.M.2.2.C and OV1057 contain different versions of E1A-FMDV 2A-E1B 55k cassettes under the transcriptional control of the E2F-1 promoter. By way of background, E1A is a transcription unit which consists of five alternatively spliced messages for the five encoded related proteins (commonly referred to as 13S, 12S, 11S, 10S, and 9S) (Shenk, T. (1996) Fundamental Virology, pp. 979-1016). Except for 9S, these proteins share the same N- and C-terminal ends. As part of the ribosomal ‘skip’, FMDV 2A remains attached to the upstream protein, as a ‘tail’, in a given cassette. Therefore, the amino acids corresponding to the 2A sequence will be added to the C-terminus of each of the fourE1A proteins, 13S, 12S, 11S, and 10S.

For E1B, two proteins (E1B 19 kD and 55 kD) are expressed via alternative splicing. Again, the cotranslational processing mediated by FMDV 2A necessitates a single, long open reading frame. Therefore, the 19k coding region of E1 B was deleted (such that the initiation codon for the 55k coding region is adjacent and in frame with the FMDV 2A terminus) in order to preserve a single open reading frame in the multicistronic cassette.

With respect to the FMDV 2A oligopeptide, previous studies indicate that its major processing activity can be localized to a 19 amino acid region at its c-terminus. Since the FMDV 2A processing activity is thought to occur by a novel ‘skip’ (between the terminal G and P of the conserved sequence -NPGP-), we sought to determine if differences in spacing of the c-terminus of FMDV 2A to the initiation codon of the second gene affected the observed processing activity. Two different C-terminal ends for the FMDV 2A oligopeptide were designed in order to examine any potential differences in efficiency in generation of the second translation product. In OV1057, the conserved amino acid sequence -NPGP- was placed directly adjacent to the initiation codon for E1B 55K. In OV1054.11.M.2.2.C, two additional amino acids (V,D) were introduced in between the -NPGP- sequence and the initiation codon of E1B 55k.

Several similar vectors, expressing one or both of the E1B proteins through mechanisms other than 2A, were used for comparison. OV945 includes the ECMV IRES to mediate translation of E1B 55k while expression of 19k is abolished via a deletion in its coding region. OV1056 and OV1054.11.M.1.1.B both retain the native E1B promoter. In OV1056, the 19k coding region has been completely removed by deletion of the region that does not overlap with 55k. As such, the initiation codon of 55k occupies the position formerly occupied by that of 19k. No such deletion was made in OV1054.11.M.1.1.B resulting in a vector identical to wild-type Ad5 with the exception of the heterologous E2F-1 promoter.

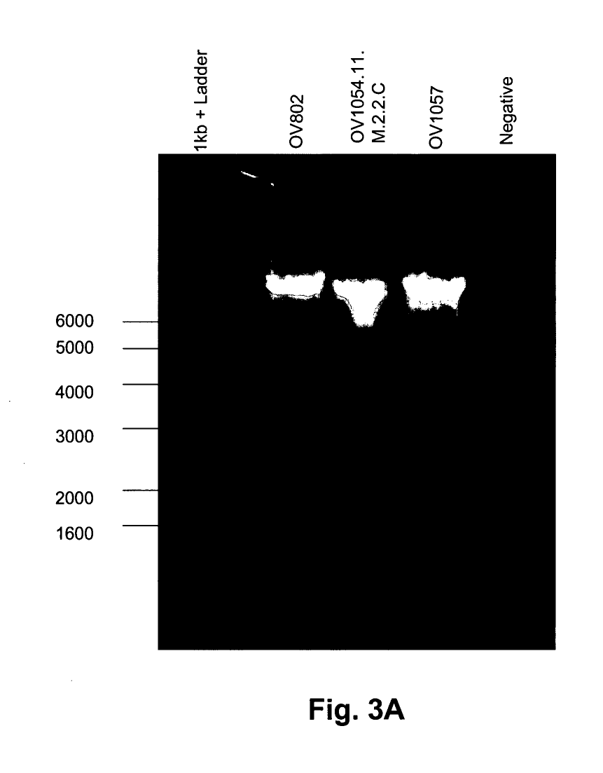

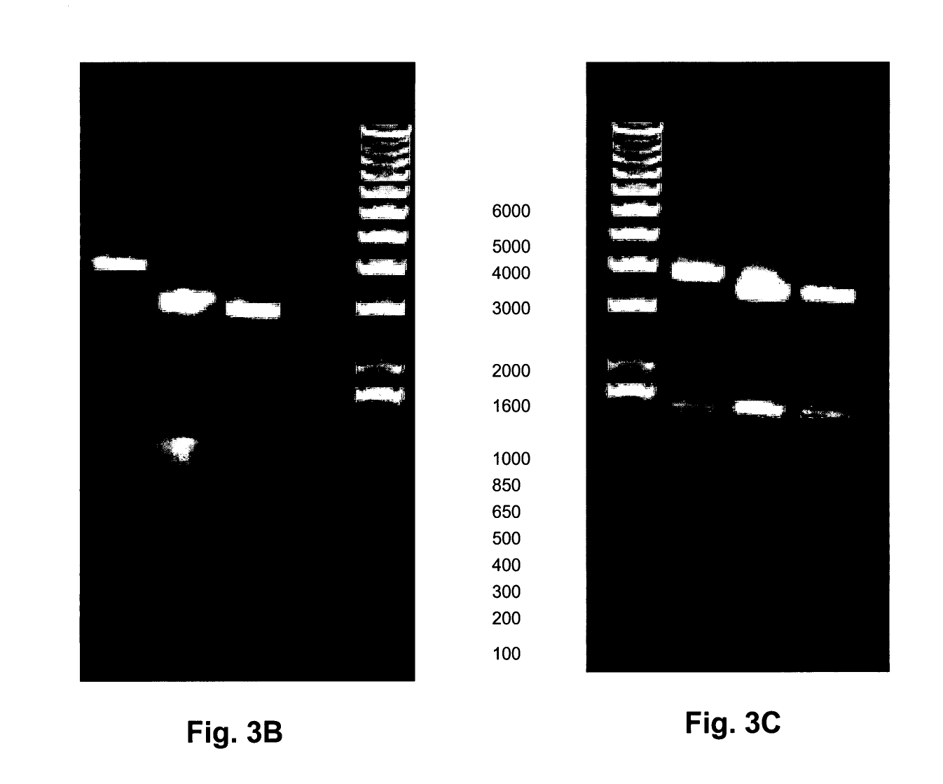

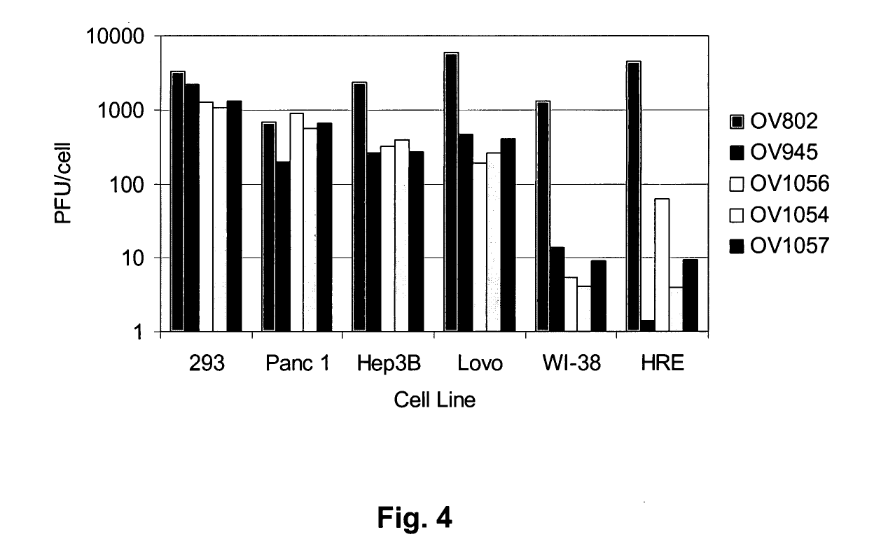

In this study, viruses were generated by homologous recombination (in 293 cells) of the left and right hand sides of a recombinant adenovirus genome. Following isolation and multiple passages of each vector through A549 cells, stocks were amplified in A549 cells, and the structures of each were verified. For this, large PCR amplicons were generated covering the modified regions of each viral genome, using primers specific for sequences in the wild-type adenovirus 5 genome (FIG. 3A). Because the modifications to each genome fall within the amplified regions, amplicon size should vary slightly. Additionally, amplicons were mapped with restriction enyzmes for which the site usage would be dependent on the particular modifications to the genome. In this case, BstX I and Acc I digests yield distinct, readily identifiable digestion patterns when applied to each of the amplicons (FIGS. 3B and 3C). The resulting digestion patterns of each vector were as predicted.

Virus replication was evaluated in a panel of both tumor and normal cell lines to examine the production of viral progeny from each of the vectors. Cells were infected at an MOI of 2 and then harvested at 72 hours post-infection. Cell lysates were prepared and used to infect 293 cells, a cell line that provides trans-complementation for adenovirus replication. Because these oncolytic vectors are driven by the tumor-selective E2F-1 promoter, it is predicted that viral production would be much lower in normal cells compared to the non-selective wild type virus, OV802. Indeed, replication of OV1054.11.M.2.2.C and OV1057 was attenuated, relative to wild-type, in the normal cell lines WI-38 and HRE by approximately 140 to 320 fold and 490 to 1160 fold, respectively (Table 3).

TABLE 3
VIRUS YIELD ASSAY (PFU/CELL)

| | 293 | Panc 1 | Hep3B | Lovo | WI-38 | HRE |
|---|---|---|---|---|---|---|
| OV802 | 3260 | 684 | 2316 | 6000 | 1300 | 4550 |
| OV945 | 2200 | 194 | 255 | 470 | 13.6 | 1.39 |
| OV1056 | 1270 | 900 | 314 | 190 | 5.4 | 62 |
| OV1054 | 1064 | 550 | 390 | 260 | 4.06 | 3.92 |
| OV1057 | 1320 | 650 | 270 | 400 | 9.14 | 9.34 |

The other E2F-transcriptionally regulated vectors, OV945 and OV1056, were similarly attenuated. When comparing the viral yield, in tumor cells, among the vectors that express E1B 55K using exogenous mechanisms to one that uses the native E1B promoter, the amount of progeny virus produced only varied by approximately 1-4 fold. These results show that FMDV 2A-meidated translation does not significantly alter the selectivity of replication seen with E2F-controlled viruses in which E1B expression is mediated by the ECMV IRES or the native E1B promoter.

As previously noted, the use of FMDV 2A to mediate the translational processing of multicistronic cassettes offers potential advantages in terms of increased expression of downstream cistrons. To determine the expression levels of E1A and E1B 55k from OV1054.11.M.2.2.C and OV1057 relative to the series of related vectors, A549 cells were separately infected with each virus at an MOI of 10. Twenty-four hours post-infection, cells were harvested and lysates prepared (as described above). Western blots were then performed to determine the expression of the E1A and E1B 55k proteins.

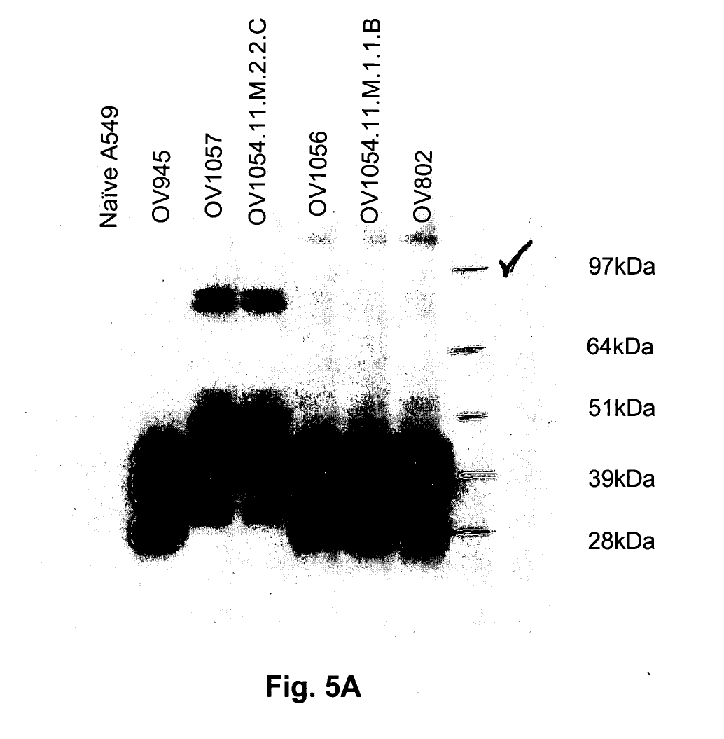

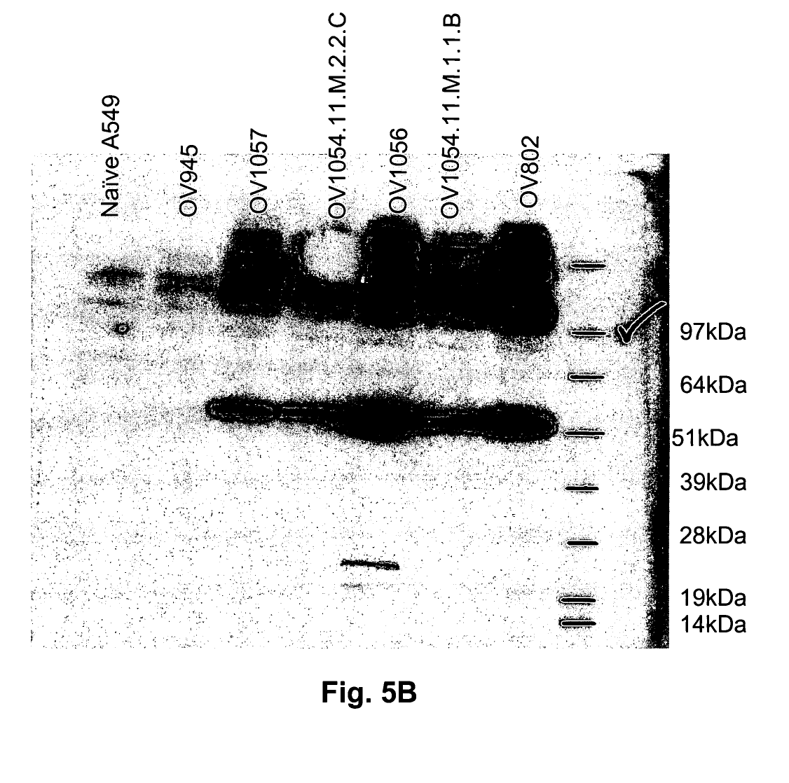

The expression of E1A was monitored for affects of the downstream element used for E1B 55K expression. The vectors containing FMDV 2A appear to synthesize reduced relative levels when compared to that produced by vectors with a wild type E1B promoter or the IRES (FIG. 5A). In lysates from OV1054.11.M.2.2.C and OV1057, E1A proteins were not detected at the positions that would be expected were they to be expressed in an unaltered form (that is, lacking an FMDV 2A ‘tail’). Instead, an equivalent number of species were detected at positions shifted upward by approximately 3 kD, roughly matching the predicted size of FMDV 2A, 2.7 kD. Notably, two larger bands are visible around the 100 kD range. They likely represent the 97 kD and 104 kD E1A-FMDV 2A-E1B 55k polypeptides that would result from the translation of unprocessed polycistronic mRNAs containing different splice variants of E1A. Although visible, they appear to be a small minority of the total detectable species. This is indicative of the relative efficiency of FMDV 2A-mediated processing and confirms previously published reports using other expression systems.

Expression of E1B 55k was determined by Western blot analysis. The expression of 55K from OV945-infected cells was barely detectable by Western blot. This could represent either the limit of detection of a non-quantitative assay. Alternatively, the inefficient gene expression from an IRES has previously been demonstrated suggesting a predictable result. In contrast, the expression of 55K from the E1A-FMDV 2A-E1B 55k vectors, OV1054.11.M.2.2.C and OV1057, was higher compared to that of OV945 (FIG. 5B) indicating that expression of the downstream gene from the cassettes including the FMDV 2A sequence was more efficient than from the cassettes including an IRES. Considering the restrictions on the packaging capacity of adenovirus vectors, the more efficient 2A system also offers the advantage of being smaller in size.

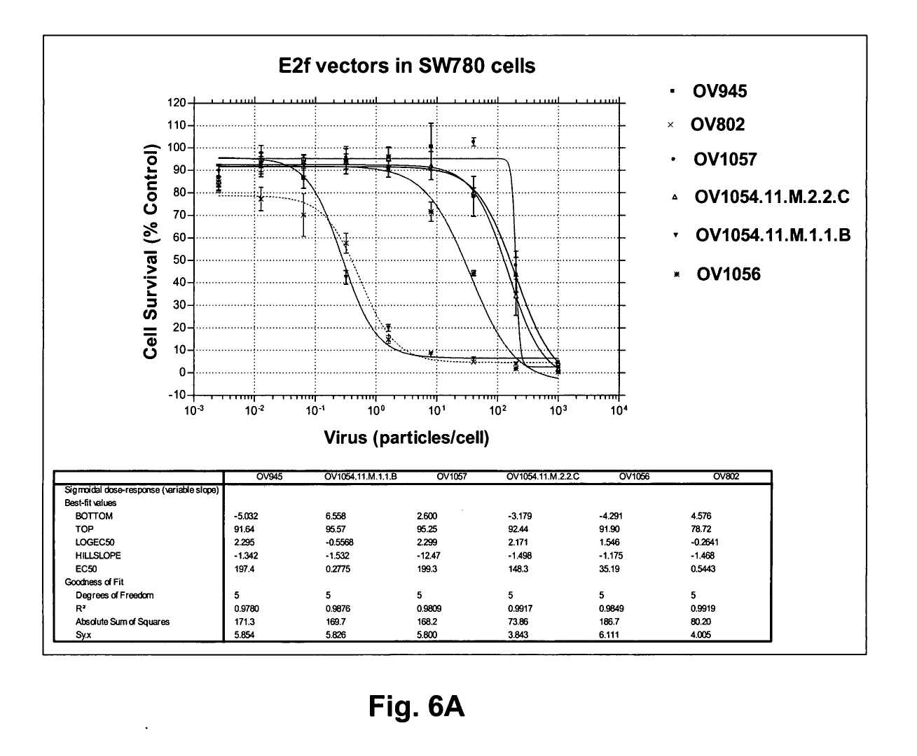

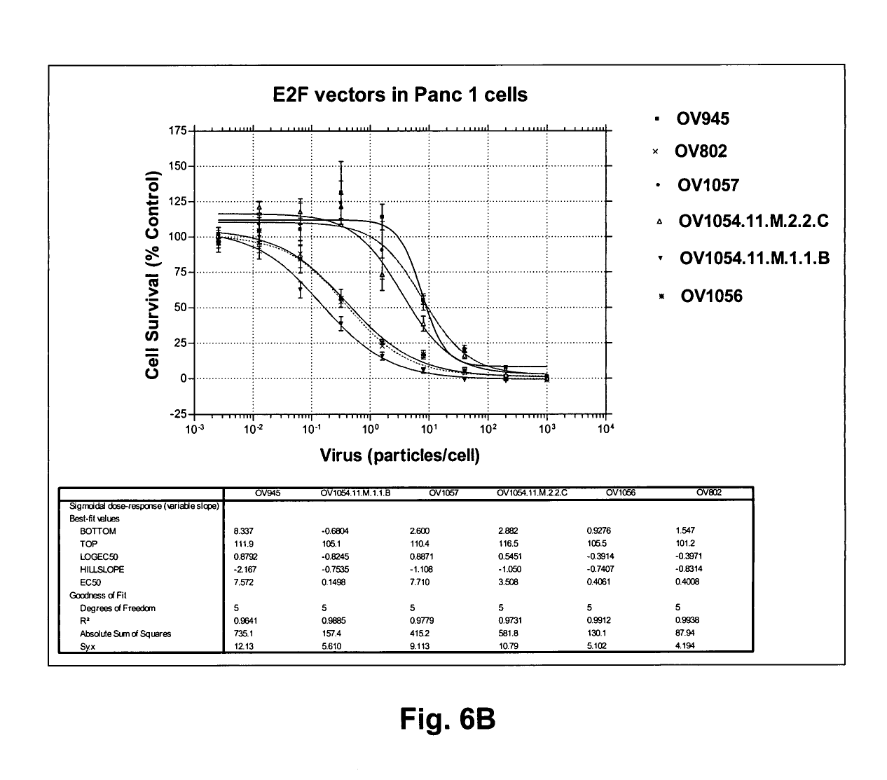

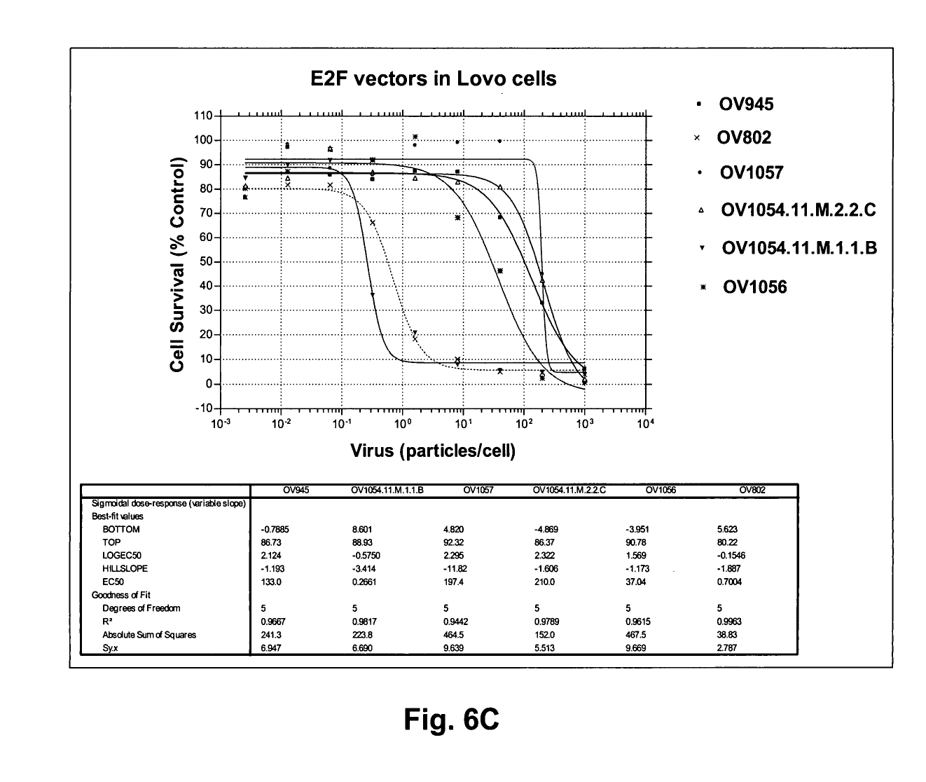

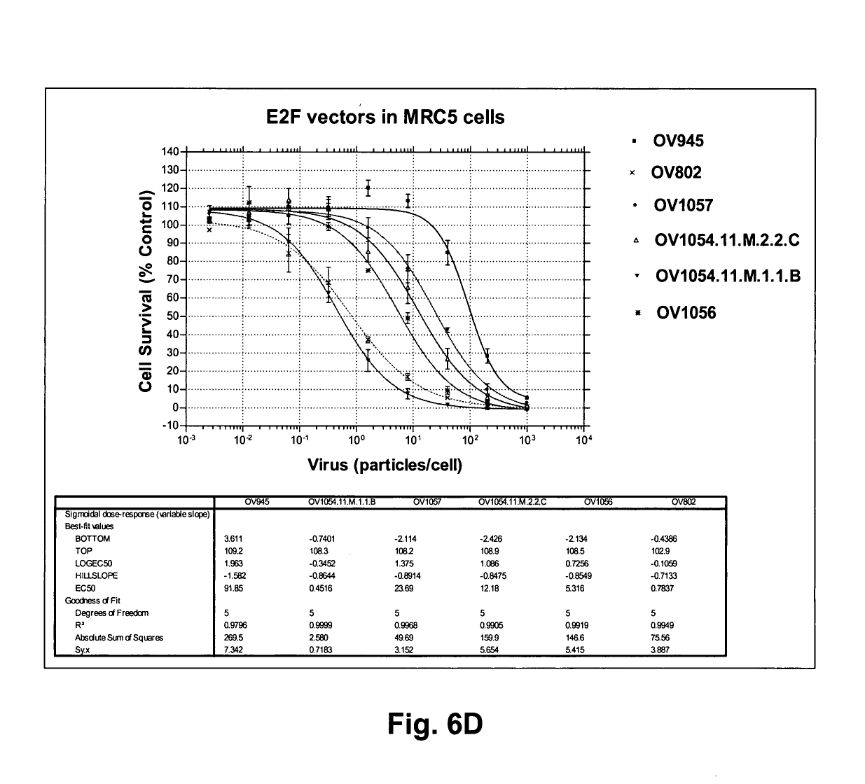

The cytotoxicity of OV1054.11.M.2.2.C and OV1057 was compared to that of the other E2F-controlled viruses as well as OV802 using an MTS assay, performed as described above. A panel of cells was separately infected with each virus over a range of concentrations. Viability of tumor cells was quantified on day seven, relative to the uninfected control. The results are shown in FIGS. 6A, 6B and 6C). Viability of normal cells was quantified on day 10, again relative to the uninfected control (FIG. 6D). Among the cell lines tested, the E1B promoter- and 19K ORF-containing vectors, wild type OV802 and OV1054.11.M.1.1.B, generally showed the most potent response, having the lowest EC50 values (the concentration of virus that kills 50% of the cell population), relative to the other vectors. The other four vectors exhibited similar degrees of viral killing. The results obtained with Panc I cells were an exception in that OV1056 was similar in toxicity to OV802 and OV1054.11.M.1.1.B.

The results provided herein show that the use of a self processing cleavage sequence such as a 2A sequence can be used to generate a functional oncolytic virus which exhibits similar selectivity of replication to other viruses in which E1B expression is mediated by an IRES or the native E1B promoter, can be used to express multiple proteins, in this case E1A and E1B 55K, and wherein cytotoxicity and viral yield are not affected. The results therefore confirm that use of a self processing cleavage sequence such as a 2A sequence is a viable option for oncolytic vectors to achieve efficient protein expression. Use of a self processing cleavage sequence such as a 2A sequence as compared to standard vector construction which relies on use of an IRES or second promoter. The small size of the 2A coding sequence makes its use advantageous in replication competent adenovirus which have a limited packing capacity for coding and regulatory sequences. In addition, elimination of the need for dual promoters reduces promoter interference that may result in reduced and/or impaired levels of expression for each coding sequence and elimination of the need for an IRES results in significantly greater expression of the coding sequence for a second protein under transcriptional control of a single TRE.

TABLE 4

SEQUENCE TABLE (FOR SEQUENCE LISTING)

| SEQ | | |
|---|---|---|
| ID | | |
| NO: | SEQUENCE | DESCRIPTION |
| 1 | LLNFDLLKLAGDVESNPGP | FMDV 2A amino |
| | | acid sequence |
| 2 | TLNFDLLKLAGDVESNPGP | FMDV 2A amino |
| | | acid sequence |
| 3 | LLKLAGDVESNPGP | Exemplary self |
| | | processing |
| | | site amino |
| | | acid sequence |
| 4 | NFDLLKLAGDVESNPGP | Exemplary self |
| | | processing |
| | | site amino |
| | | acid sequence |
| 5 | QLLNFDLLKLAGDVESNPGP | Exemplary self |
| | | processing |
| | | site amino |
| | | acid sequence |
| 6 | APVKQTLNFDLLKLAGDVESN | Exemplary self |
| | PGP | processing |
| | | site amino |
| | | acid sequence |
| 7 | VTELLYRMKRAETYCPRPLLA | Exemplary self |
| | IHPTEARHKQKIVAPVKQTLN | processing |
| | FDLLKLA GDVESNPGP | site amino |
| | | acid sequence |
| 8 | LLAIHPTEARHKQKIVAPVKQ | Exemplary self |
| | TLNFDLLKLAGDVESNPGP | processing |
| | | site amino |
| | | acid sequence |
| 9 | EARHKQKIVAPVKQTLNFDLL | Exemplary self |
| | KLAGDVESNPGP | processing |
| | | site amino |
| | | acid sequence |
| 10 | furin cleavage site | Exemplary |
| | with the consensus | additional |
| | sequence RXK(R)R | proteolytic |
| | | cleavage site |
| | | amino acid |
| | | sequence |
| 11 | furin cleavage site | Exemplary |
| | RAKR | additional |
| | | proteolytic |
| | | cleavage site |
| | | amino acid |
| | | sequence |
| 12 | Factor Xa cleavage | Exemplary |
| | site: IE(D)GR | additional |
| | | proteolytic |
| | | cleavage site |
| | | amino acid |
| | | sequence |
| 13 | Signal peptidase I | Exemplary |
| | cleavage site: e.g. | additional |
| | LAGFATVAQA | proteolytic |
| | | cleavage site |
| | | amino acid |
| | | sequence |
| 14 | Thrombin cleavage | Exemplary |
| | site: LVPRGS | additional |
| | | proteolytic |
| | | cleavage site |
| | | amino acid |
| | | sequence |
| 15 | TGGTACCATCCGGACAAAGCC | 270 bp |
| | TGCGCGCGCCCCGCCCCGCCA | fragment |
| | TTGGCCGT | containing |
| | ACCGCCCCGCGCCGCCGCCCC | sequences from |
| | ATCCCGCCCCTCGCCGCCGGG | the human E2F |
| | TCCGGCGC | promoter |
| | GTTAAAGCCAATAGGAACCGC | |
| | CGCCGTTGTTCCCGTCACGGC | |
| | CGGGGCAG | |
| | CCAATTGTGGCGGCGCTCGGC | |
| | GGCTCGTGGCTCTTTCGCGGC | |
| | AAAAAGGA | |
| | TTTGGCGCGTAAAAGTGGCCG | |
| | GGACTTTGCAGGCAGCGGCGG | |
| | CCGGGGGC | |
| | GGAGCGGGATCGAGCCCTCG | |
| 16 | ACCGTGGCGGAGGGACTGGGG | 241 bp |
| | ACCCGGGCACCCGTCCTGCCC | fragment |
| | CTTCACCTTCCAGCTCCGCCT | containing |
| | CCTCCGCGCGGACCCCGCCCC | sequences from |
| | GTCCCGACCCCTCCCGGGTCC | the human |
| | CCGGCCCAGCCCCCTCCGGGC | telomerase |
| | CCTCCCAGCCCCTCCCCTTCC | (TERT) |
| | TTTCCGCGGCCCCGCCCTCTC | promoter |
| | CTCGCGGCGCGAGTTTCAGGC | |
| | AGCGCTGCGTCCTGCTGCGCA | |
| | CGTGGGAAGCCCTGGCCCCGG | |
| | CCACCCCCGC | |
| 17 | CCCCACGTGGCGGAGGGACTG | 245 bp |
| | GGGACCCGGGCACCCGTCCTG | fragment |
| | CCCCTTCA | containing |
| | CCTTCCAGCTCCGCCTCCTCC | sequences |
| | GCGCGGACCCCGCCCCGTCCC | from the |
| | GACCCCTCC | human |
| | CGGGTCCCCGGCCCAGCCCCC | TERT promoter |
| | TCCGGGCCCTCCCAGCCCCTC | |
| | CCCTTCCTT | |
| | TCCGCGGCCCCGCCCTCTCCT | |
| | CGCGGCGCGAGTTTCAGGCAG | |
| | CGCTGCGTC | |
| | CTGCTGCGCACGTGGGAAGCC | |
| | CTGGCCCCGGCCACCCCCGCG | |

1. A cytolytic replication competent adenovirus vector comprising in sequential order: a left ITR, a heterologous transcriptional regulatory element (TRE) operably linked to the coding sequence for a first adenoviral gene, a sequence encoding a self-processing cleavage site, the coding sequence for a second adenoviral gene and a right ITR.

2. The adenovirus vector according to claim 1, wherein said first adenoviral gene is an adenoviral gene essential for replication.

3. The adenovirus vector according to claim 2, wherein said adenoviral gene essential for replication is an early gene.

4. The adenovirus vector according to claim 3, wherein said adenoviral gene essential for replication is selected from the group consisting of E1A, E1B, E2 and E4.

5. The adenovirus vector according to claim 4, wherein said adenoviral gene essential for replication is E1A or E1B.

6. The adenovirus vector according to claim 5, wherein E1A or E1B has a mutation in, or deletion of its endogenous promoter.

7. The adenovirus vector according to claim 2, wherein said adenoviral gene essential for replication is a late gene.

8. An adenovirus vector according to claim 1, wherein said second adenoviral gene is an adenoviral gene essential for replication.

9. The adenovirus vector according to claim 8, wherein said adenoviral gene essential for replication is an early gene.

10. The adenovirus vector according to claim 9, wherein said adenoviral gene essential for replication is selected from the group consisting of E1A, E1B, E2 and E4.

11. The adenovirus vector according to claim 10, wherein said adenoviral gene essential for replication is E1A or E1B.

12. The adenovirus vector according to claim 11, wherein E1A or E1B has a mutation in or deletion of its endogenous promoter.

13. The adenovirus vector according to claim 7, wherein said adenoviral gene essential for replication is a late gene.

14. An adenovirus vector according to claim 4, wherein the sequence encoding said self-processing cleavage site comprises a 2A sequence.

15. An adenovirus vector according to claim 14, wherein said 2A sequence is a Foot and Mouth Disease Virus (FMDV) sequence.

16. An adenovirus vector according to claim 15, wherein said 2A sequence encodes an oligopeptide comprising amino acid residues LLNFDLLKLAGDVESNPGP (SEQ ID NO:1) or TLNFDLLKLAGDVESNPGP (SEQ ID NO:2).

17. An adenovirus vector according to claim 15, further comprising an additional proteolytic cleavage site wherein said cleavage site is a furin cleavage site with the consensus sequence RXK(R)R (SEQ ID NO:10).

18. An adenovirus vector according to claim 4, wherein said heterologous TRE comprises a promoter selected from the group consisting of a tissue-specific promoter, a tumor-specific promoter, a developmental stage-specific promoter and a cell status specific promoter.

19. An adenovirus vector according to claim 18, wherein said heterologous TRE further comprises an enhancer.

20. An adenovirus vector according to claim 4, wherein said heterologous TRE is a selected from the group consisting of an E2F responsive promoter, a TERT promoter, a prostate-specific antigen (PSA) transcriptional regulatory element (PSA-TRE), a probasin transcriptional regulatory element (PB-TRE), a human glandular kallikrein transcriptional regulatory element (HKLK2-TRE), a carcinoembryonic antigen transcriptional regulatory element (CEA-TRE), an alpha-fetoprotein transcriptional regulatory element (AFP-TRE), a uroplakin II transcriptional regulatory element (UPII-TRE); a PRL-3 transcriptional regulatory element TRE (PRL-3 TRE); a melanocyte cell-specific transcriptional response element (melanocyte TRE) and a CRG-L2 transcriptional regulatory element (CRG-L2 TRE).

21. An adenovirus vector according to claim 20, wherein said heterologous TRE is an E2F responsive promoter.

22. An adenovirus vector according to claim 21, wherein said E2F responsive promoter comprises SEQ ID NO:15.

23. An adenovirus vector according to claim 20, wherein said heterologous TRE is a TERT promoter.

24. An adenovirus vector according to claim 23, wherein said TERT promoter is a human TERT promoter.

25. An adenovirus vector according to claim 24, wherein said TERT promoter comprises SEQ ID NO:16 or SEQ ID NO:17.

26. An adenovirus vector according to claim 5, wherein the EIB gene has a deletion of the 19-kDa region.

27. An adenovirus vector according to claim 4, further comprising a mutation or deletion in an E3 coding region.

28. An adenovirus vector according to claim 27, wherein at least one E3 coding region has been deleted from said backbone.

29. An adenovirus vector according to claim 4, wherein the E3 region in said backbone codes for at least one of the native E3 proteins.

30. An adenovirus vector according to claim 29, wherein said E3 coding region is selected from the group consisting of E3-6.7, KDa, gp19 KDa, 11.6 KDa (ADP), 10.4 KDa (RIDα), 14.5 KDa (RIDβ), and E3-14.7 Kda.

31. An adenovirus vector according to claim 29 wherein the E3 region in said backbone codes for all of the native E3 proteins.

32. An isolated host cell comprising the adenovirus vector of claim 15.

33. An isolated host cell comprising the adenovirus vector of claim 22.

34. An isolated host cell comprising the adenovirus vector of claim 25.

35. A composition comprising a replication-competent adenovirus vector according to claim 15 and a pharmaceutically acceptable excipient.

36. A composition comprising a replication-competent adenovirus vector according to claim 22 and a pharmaceutically acceptable excipient.

37. A composition comprising a replication-competent adenovirus vector according to claim 25 and a pharmaceutically acceptable excipient.

38. A cytolytic replication competent adenovirus vector comprising in sequential order: a left ITR, heterologous transcriptional regulatory element (TRE) operably linked to the coding sequence for an adenoviral gene, a sequence encoding a self-processing cleavage site, the coding sequence for a transgene and a right ITR.

39. An adenovirus vector according to claim 38, wherein said adenoviral gene is an adenoviral gene essential for replication.

40. The adenovirus vector of claim 39, wherein said adenoviral gene essential for replication is an early gene.

41. The adenovirus vector of claim 40, wherein said adenoviral gene essential for replication is selected from the group consisting of E1A, E1B, E2 and E4.

42. The adenovirus vector of claim 41, wherein said adenoviral gene essential for replication is E1A or E1B.

43. The adenovirus vector of claim 42, wherein E1A or E1B has a mutation in or deletion of its endogenous promoter.

44. The adenovirus vector of claim 39, wherein said adenoviral gene essential for replication is a late gene.

45. An adenovirus vector according to claim 41, wherein said transgene is a cytotoxic gene.

46. The adenovirus vector of claim 45, wherein said cytotoxic gene is an adenoviral death protein (ADP) gene.

47. An adenovirus vector according to claim 41, wherein said transgene is GM-CSF.

48. An adenovirus vector according to claim 41, wherein the sequence encoding said self-processing cleavage site comprises a 2A sequence.

49. An adenovirus vector according to claim 48, wherein said 2A sequence is a Foot and Mouth Disease Virus (FMDV) sequence.

50. An adenovirus vector according to claim 49, wherein said 2A sequence encodes an oligopeptide comprising amino acid residues LLNFDLLKLAGDVESNPGP (SEQ ID NO:1) or TLNFDLLKLAGDVESNPGP (SEQ ID NO:2).

51. An adenovirus vector according to claim 41, further comprising an additional proteolytic cleavage site is a furin cleavage site with the consensus sequence RXK(R)R (SEQ ID NO:10).

52. An adenovirus vector according to claim 41, wherein said heterologous TRE comprises a promoter selected from the group consisting of a tissue-specific, a tumor-specific, a developmental stage-specific and a cell status specific promoter.

53. An adenovirus vector according to claim 52, wherein said heterologous TRE further comprises an enhancer.

54. An adenovirus vector according to claim 41, wherein said heterologous TRE is a selected from the group consisting of an E2F responsive promoter, a TERT promoter, a prostate-specific antigen (PSA) transcriptional regulatory element (PSA-TRE), a probasin transcriptional regulatory element (PB-TRE), a human glandular kallikrein transcriptional regulatory element (HKLK2-TRE), a carcinoembryonic antigen transcriptional regulatory element (CEA-TRE), an alpha-fetoprotein transcriptional regulatory element (AFP-TRE), a uroplakin II transcriptional regulatory element (UPII-TRE); a PRL-3 transcriptional regulatory element TRE (PRL-3 TRE); a melanocyte cell-specific transcriptional response element (melanocyte TRE) and a CRG-L2 transcriptional regulatory element (CRG-L2 TRE).

55. An adenovirus vector according to claim 54, wherein said heterologous TRE is an E2F responsive promoter.

56. An adenovirus vector according to claim 55, wherein said E2F responsive promoter comprises SEQ ID NO:15.

57. An adenovirus vector according to claim 41, wherein said heterologous TRE is a TERT promoter.

58. An adenovirus vector according to claim 57, wherein said TERT promoter is a human TERT promoter.

59. An adenovirus vector according to claim 58, wherein said TERT promoter comprises SEQ ID NO:16 or SEQ ID NO:17.

60. An adenovirus vector according to claim 43, wherein the E1B gene has a deletion of the 19-kDa region.

61. An adenovirus vector according to claim 41, further comprising a mutation or deletion in an E3 coding region.

62. An adenovirus vector according to claim 61, wherein at least one of the E3 coding regions have been deleted from said backbone.

63. An adenovirus vector according to claim 41, wherein the E3 region in said backbone codes for at least one of the native E3 proteins.

64. An adenovirus vector according to claim 63, wherein said E3 coding region is selected from the group consisting of E3-6.7, KDa, gp19KDa, 11.6 KDa (ADP), 10.4 KDa (RIDα), 14.5 KDa (RIDβ), and E3-14.7 Kda.

65. An adenovirus vector according to claim 63, wherein the E3 region in said backbone codes for all of the native E3 proteins.

66. An isolated host cell comprising the adenovirus vector of claim 49.

67. An isolated host cell comprising the adenovirus vector of claim 56.

68. An isolated host cell comprising the adenovirus vector of claim 59.

69. A composition comprising a replication-competent adenovirus vector according to claim 49 and a pharmaceutically acceptable excipient.

70. A composition comprising a replication-competent adenovirus vector according to claim 56 and a pharmaceutically acceptable excipient.

71. A composition comprising a replication-competent adenovirus vector according to claim 59 and a pharmaceutically acceptable excipient.


SEQUENCE LISTING (17 sequences)

SEQ ID NO: 1 - protein, 19 aa, Artificial Sequence
  FMDV 2A
LLNFDLLKLAGDVESNPGP

SEQ ID NO: 2 - protein, 19 aa, Artificial Sequence
  FMDV 2A
TLNFDLLKLAGDVESNPGP

SEQ ID NO: 3 - protein, 14 aa, Artificial Sequence
  2A variant
LLKLAGDVESNPGP

SEQ ID NO: 4 - protein, 17 aa, Artificial Sequence
  2A variant
NFDLLKLAGDVESNPGP

SEQ ID NO: 5 - protein, 20 aa, Artificial Sequence
  2A variant
QLLNFDLLKLAGDVESNPGP

SEQ ID NO: 6 - protein, 24 aa, Artificial Sequence
  2A variant
APVKQTLNFDLLKLAGDVESNPGP

SEQ ID NO: 7 - protein, 58 aa, Artificial Sequence
  2A variant
VTELLYRMKRAETYCPRPLLAIHPTEARHKQKIVAPVKQTLNFDLLKLAGDVESNPGP

SEQ ID NO: 8 - protein, 40 aa, Artificial Sequence
  2A variant
LLAIHPTEARHKQKIVAPVKQTLNFDLLKLAGDVESNPGP

SEQ ID NO: 9 - protein, 33 aa, Artificial Sequence
  2A variant
EARHKQKIVAPVKQTLNFDLLKLAGDVESNPGP

SEQ ID NO: 10 - protein, 5 aa, Artificial Sequence
  exemplary additional proteolytic cleavage site
RXKRR

SEQ ID NO: 11 - protein, 4 aa, Artificial Sequence
  exemplary additional proteolytic cleavage site
RAKR

SEQ ID NO: 12 - protein, 5 aa, Artificial Sequence
  exemplary additional proteolytic cleavage site
IEDGR

SEQ ID NO: 13 - protein, 10 aa, Artificial Sequence
  exemplary additional proteolytic cleavage site
LAGFATVAQA

SEQ ID NO: 14 - protein, 6 aa, Artificial Sequence
  exemplary additional proteolytic cleavage site
LVPRGS

SEQ ID NO: 15 - DNA, 270 bp, Homo sapiens
tggtaccatccggacaaagcctgcgcgcgccccgccccgccattggccgtaccgccccgcgccgccgccccatcccgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcg

SEQ ID NO: 16 - DNA, 241 bp, Homo sapiens
accgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgc

SEQ ID NO: 17 - DNA, 245 bp, Homo sapiens
ccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg